(19) United States
(12) Patent Application Publication
(10) Pub. No.: US 2025/0152687 A1
(43) Pub. Date: May 15, 2025

(54) CONSENSUS SEQUENCE OF THE ANTIGEN TELOMERASE AND THE USE THEREOF IN PREVENTIVE AND THERAPEUTIC VACCINATION

(71) Applicant: EVVIVAX S.R.L., Roma (IT)

(72) Inventors: Luigi AURISICCHIO, Roma (IT); Antonella CONFORTI, Roma (IT)

(21) Appl. No.: 18/018,541

(22) PCT Filed: Jul. 26, 2021
(86) PCT No.: PCT/IT2021/050227

§ 371 Date: Jan. 27, 2023

(30) Foreign Application Priority Data
Jul. 29, 2020 (IT) 102020000018379

(51) Int. Cl. A61K 39/00; A61P 35/00; C12N 9/12

(52) U.S. Cl. CPC A61K 39/001157; A61P 35/00; C12N 9/1276; A61K 2039/53; A61K 2039/572; C07K 2319/30; C07K 2319/55; C12Y 207/07049

(57) ABSTRACT

A consensus sequence of the antigen telomerase (ConTRt) can be generated and used in preventive and therapeutic vaccination. The consensus sequence of telomerase was generated by the fusion of two sequences, one belonging to human telomerase (hTERT) and the other to dog telomerase (dTERT), with the aim of developing an effective vaccine for the treatment of tumors expressing both human and dog telomerase, hence in both the human and veterinary sectors.

### PRIORITY AND CROSS REFERENCE TO RELATED APPLICATIONS

This application is the U.S. National Phase Application under 35 U.S.C. § 371 of International Application No. PCT/IT2021/050227, filed Jul. 26, 2021, and published as WO2022/024156A2 on Feb. 3, 2022, which claims the benefit of Foreign Application No. IT102020000018379, filed Jul. 29, 2020. Any and all applications for which a foreign or a domestic priority is claimed is/are identified in the Application Data Sheet filed herewith and is/are hereby incorporated by reference in their entireties under 37 C.F.R. § 1.57.

### REFERENCE TO SEQUENCE LISTING

The present application is being filed along with a Sequence Listing in electronic format. The Sequence Listing is provided as a file entitled SequenceListing BARZ070.001APC.txt, created Jan. 26, 2023, which is approximately 84 kilobytes in size, which is replaced by a Replacement Electronic Sequence Listing submitted as herewith a file entitled BARZ070.001ACPCorrectedSequenceListing.txt, which is 76,442 bytes in size and was created on Jul. 17, 2024. The information in the electronic format of the Sequence Listing is incorporated herein by reference in its entirety.

### FIELD

The present invention relates to a consensus sequence of the antigen telomerase and the use thereof in preventive and therapeutic vaccination. In particular, the present invention relates to the generation of a consensus sequence of the antigen telomerase (ConTRt) and the use thereof in preventive and therapeutic vaccination, wherein the telomerase consensus sequence was generated by the fusion of two sequences, one belonging to human telomerase (hTERT) and the other to dog telomerase (dTERT), with the aim of developing an effective vaccine for the treatment of tumours expressing both human and dog telomerase, hence in both the human and veterinary sectors.

### BACKGROUND

It is well known that vaccination is a powerful tool for obtaining the activation of the immune system against pathogens of every type. Vaccination represents the first-line preventive intervention for eliminating the risk of contracting dangerous infectious diseases which can spread throughout a large part of the population, at times causing true epidemics. At present, thanks to vaccines it is possible to defeat highly dangerous infectious diseases for which a therapy either does not exist (poliomyelitis and hepatitis B) or is not always effective (diphtheria, tetanus) or diseases that can be a cause of serious complications (measles, German measles and whooping cough).

Immunotherapy, i.e. the therapeutic treatment of disease by means of an activation or suppression of the immune system, has demonstrated to be effective also against various types of tumours, despite having to overcome several limitations, such as immunological tolerance and low immunogenicity. The objective of tumour immunotherapy is therefore to restore the ability of the immune system to recognise tumour cells and eliminate them effectively, by overcoming the mechanisms whereby tumours suppress the immune response. To this end, the immune system recognises tumour-associated molecules (tumour-associated antigens) [Liu 2017], which, despite also being expressed by normal cells, appear on tumour cells in an abnormal manner in terms of quantity, site or time (for example, the carcinoembryonic antigen CEA) or molecules expressed exclusively by tumour cells (tumour-specific antigens), such as the products of normally silent genes [Seremet 2011]. Notwithstanding the discovery of tumour antigens as a target of immunotherapeutic strategies, the greatest obstacle in the development of cancer immunotherapy has been the absence of an antigen common to the majority of patients affected by the most common types of cancer, and thus different research groups have concentrated on the search for a universal tumour-associated antigen capable of inducing a T-cell response of a cytotoxic type (CTL) against a wide range of tumour types.

The tumour-associated antigen telomerase represents an ideal candidate for anti-tumour immunotherapy. Telomerase is an enzyme responsible for the lengthening of DNA telomeres, a phenomenon typical of actively proliferating cells, such as embryonal ones, and which is by contrast absent under physiological conditions in somatic cells. Under certain pathological conditions, in which proliferative activity increases, such as in neoplasms, telomerase activity also increases enormously, and various studies have by now demonstrated the correlation existing between telomerase activity and cancer [Akincilar 2016]. In addition to being expressed in about 90% of tumour types, telomerase is degraded by cellular proteasomes into peptides then presented in the context of MHC class I on the surface of the tumour cell as the target of the activity of antigen-specific cytotoxic T-cells. Considering the considerably lower levels of telomerase activity in somatic cells under physiological conditions, the number of specific MHC/TERT peptide complexes present at the level of the plasma membrane are insufficient to activate an immune response [Gross 2004], whereas the abnormal expression of telomerase in tumour cells enables the immune system to discriminate between normal tissue and neoplastic tissue, thus making telomerase an ideal candidate as a universal target antigen for an antitumour vaccine [Vonderheide 2002]. Furthermore, J. Yan et al., 2013, describe an optimised DNA vaccine that targets the reverse transcriptase of human telomerase, which should stimulate antitumour immunity [J. Yan et al. 2013].

Consequently, in recent years various phase I/II clinical studies have been started up in humans, with the aim of conducting experiments on antitumour vaccines targeting hTERT and based on peptides or autologous dendritic cells exhibiting the antigen telomerase [Kailashiya 2017]. However, such vaccines, despite the high immunogenicity and specificity for telomerase, are HLA-restricted and thus not potentially universal. In fact, to date such clinical studies have not yet achieved the hoped-for results, demonstrating a limited clinical impact on the majority of patients. Accordingly, in order to overcome such limitations, DNA vaccines targeting hTERT have been formulated, in consideration of the greater stability, ease of production and universal applicability of DNA [Dharmapuri 2009; Thalmensi 2016]. Furthermore, with the aim of improving their immunogenicity, new genetic vaccination methods have been conceived, e.g. electroporation, which have considerably increased the effectiveness of DNA vaccines, thus promoting experimentation therewith in phase I/II clinical studies in humans [Teixeira 2020].

In recent years, such therapeutic approaches have also been developed for the veterinary field. It is well known, in fact, that domestic animals, in particular dogs, develop tumours whose biology, incidence and response to therapies are similar to those of humans [Ranieri 2013]. Furthermore, unlike other species used as preclinical models (for example monkeys), dogs represent an excellent model 5 for studies on the effectiveness of antitumour therapies, considering the spontaneous onset and intrinsic interindividual heterogeneity of tumours, as occurs in humans. In dogs as well, furthermore, telomerase is expressed in over 90% of tumour types [Nasir 2008]. In various models of dog tumours expressing dTERT, clinical studies have thus been conducted which have demonstrated the effectiveness of genetic vaccines, in combination with chemotherapy treatment, in inducing a specific immune response to dTERT together with a greater antitumour effect and longer patient survival [Peruzzi 2010; Gavazza 2013; Thalmensi 2019]. Furthermore, also in this animal model, various strategies for administering the vaccine have been experimented with, including electroporation [Impellizeri 2012] and high pressure without the use of needles [Bergman 2006], the former method being capable of inducing a more powerful antigen-specific immune response than the latter [Yu 2011]. There are known immunogenic compositions comprising a nucleic acid which comprise a sequence encoding a cat or dog telomerase deprived of telomerase catalytic activity (WO2014/154904; WO2014/154905).

At present, only one genetic vaccine (Oncept®, distributed by Merial) directed against tyrosinase and used in the treatment of canine melanoma has been approved for sale by the USDA.

Therefore, in the light of the above, it appears evident that there is a need to provide more effective vaccination strategies which overcome the limits of the presently known methods for the treatment of telomerase-expressing tumours, in both the human and veterinary sectors.

It is well known that vaccination with a xenogeneic antigen, which shows a sufficient degree of homology with its ortholog, represents an effective method for obtaining more potent immune responses and overcoming tolerance towards self-proteins [Fattori 2009]. A particular example is offered by the xenogeneic DNA vaccination against ErbB2, a tyrosine kinase expressed in 20-30% of breast cancers and in different very aggressive types of epithelial tumours. In fact, the vaccine against the human form of ErbB2 (Her2) is capable of breaking immunological tolerance [Jacob 2006] in a model of transgenic mice with breast cancer expressing the rat form of ErbB2 (neu) due to the phenomenon of cross-reactivity [Cavallo 2014]. In particular, the autologous vaccine is capable of inducing antibodies towards the self-antigen, whereas the xenogeneic vaccine stimulates the activation of T-cells against the ortholog antigen. Consequently, in order to combine both immune system activation mechanisms, DNA sequences encoding for a chimeric rat/human or human/rat ErbB2 protein have been created, which has demonstrated to be able to induce potent responses against Her2/neu both in wild-type mice and in Her2/neu transgenic mice [Quaglino 2010]. The effectiveness of xenogeneic vaccination in breaking tolerance towards self-antigens and inducing an antitumour effect has been demonstrated in clinical studies conducted in humans [Ginsberg 2010; Eriksson 2013]. In canine melanoma as well, the strategy of xenogeneic vaccination, in particular with a plasmid encoding for the human form of the antigen CSPG4, has revealed to be effective for obtaining longer survival times in patients, who developed antibodies directed against both the human and canine forms of the target antigen [Riccardo 2014]. Therefore, xenogeneic vaccination represents a therapeutic anti-tumour strategy that may be explored against various tumour-associated antigens both in humans and in dogs.

### SUMMARY

In some embodiments, an amino acid sequence of the antigen telomerase, where the amino acid sequence being selected from the group consisting of: an amino acid consensus sequence of the antigen telomerase comprising of sequence SEQ ID NO:1; a chimeric amino acid sequence of dog and human telomerase comprising of sequence SEQ ID NO:2, or a chimeric amino acid sequence of human and dog telomerase comprising of sequence SEQ ID NO:3, preferably an amino acid consensus sequence of the antigen telomerase comprising or consisting of sequence SEQ ID NO:1. In some embodiments, the amino acid sequence also includes one or more leader sequences. In some embodiments, the amino acid sequence also includes one or more one or more immunomodulating amino acid sequences.

In some embodiments, a nucleotide sequence encodes the amino acid sequence of the antigen telomerase consisting of: an amino acid consensus sequence of the antigen telomerase comprising of sequence SEQ ID NO:1; a chimeric amino acid sequence of dog and human telomerase comprising of sequence SEQ ID NO:2, or a chimeric amino acid sequence of human and dog telomerase comprising of sequence SEQ ID NO:3, preferably an amino acid consensus sequence of the antigen telomerase comprising of sequence SEQ ID NO: 1.

In some embodiments, the nucleotide sequence is selected from the group consisting of a nucleotide sequence comprising sequence SEQ ID NO:4, or a nucleotide sequence comprising a sequence having a sequence identity of at least 80% with respect to SEQ ID NO:4, a nucleotide sequence comprising SEQ ID NO: 5 or a nucleotide sequence comprising SEQ ID NO:6. In some embodiments, the nucleic acid sequence comprises the nucleotide sequence SEQ ID NO:4, or a nucleotide sequence comprising a sequence having a sequence identity of at least 80% with respect to SEQ ID NO:4, and a nucleotide sequence encoding the profilin-like protein of Toxoplasma gondii. In some embodiments, the nucleotide sequence encoding the profilin-like protein of Toxoplasma gondii comprises SEQ ID NO:31.

In some embodiments, a nucleotide sequence encodes the amino acid sequence of the antigen telomerase consisting of: an amino acid consensus sequence of the antigen telomerase comprising of sequence SEQ ID NO:1; a chimeric amino acid sequence of dog and human telomerase comprising of sequence SEQ ID NO:2, or a chimeric amino acid sequence of human and dog telomerase comprising of sequence SEQ ID NO:3, preferably an amino acid consensus sequence of the antigen telomerase comprising or consisting of sequence SEQ ID NO:1; and one or more leader sequences. The one or more leader sequence is a secretion leader sequence of a protein selected from the group consisting of tissue plasminogen activator (TPA), IgK, growth hormone, serum albumin, and alkaline phosphatase.

In some embodiments, a nucleotide sequence encodes the amino acid sequence of the antigen telomerase consisting of: an amino acid consensus sequence of the antigen telomerase comprising of sequence SEQ ID NO:1; a chimeric amino acid sequence of dog and human telomerase comprising of sequence SEQ ID NO:2, or a chimeric amino acid sequence of human and dog telomerase comprising of sequence SEQ ID NO:3, preferably an amino acid consensus sequence of the antigen telomerase comprising or consisting of sequence SEQ ID NO:1; and one or more one or more immunomodulating amino acid sequences. The one or more immunomodulating amino acid sequences are selected from the group consisting of the fragment crystallisable (Fc) region, profilin-like protein of Toxoplasma gondii (PFTG) or a functional fragment derived therefrom, the B subunit of the heat-labile toxin of Escherichia coli (LTB) and the tetanus toxin (TT).

In some embodiments, an expression vector comprises the nucleotide sequence. In some embodiments, the expression vector is selected from the group consisting of a plasmid, an RNA, a replicating RNA, amplicons obtained by PCR, and a viral vector.

In some embodiments, a pharmaceutical composition comprises of the amino acid sequence in combination with one or more excipients and/or adjuvants.

In some embodiments, a method of vaccinating a subject against telomerase-expressing tumors or treating a subject having a telomerase-expressing tumors, comprise administering the amino acid sequence to the subject.

In some embodiments, a DNA or RNA-based vaccine comprises a nucleotide sequence. The nucleotide sequence encodes the amino acid sequence of the antigen telomerase consisting of: an amino acid consensus sequence of the antigen telomerase comprising of sequence SEQ ID NO:1; a chimeric amino acid sequence of dog and human telomerase comprising of sequence SEQ ID NO: 2, or a chimeric amino acid sequence of human and dog telomerase comprising of sequence SEQ ID NO:3, preferably an amino acid consensus sequence of the antigen telomerase comprising of sequence SEQ ID NO:1.

In some embodiments, a method of vaccinating a subject against telomerase-expressing tumors or treating a subject having a telomerase-expressing tumors, comprise administering a vaccine. The vaccine is a DNA or RNA-based vaccine which comprises a nucleotide sequence encoding the amino acid sequence of the antigen telomerase consisting of: an amino acid consensus sequence of the acid sequence of dog and human telomerase comprising of sequence SEQ ID NO: 2, or a chimeric amino acid sequence of human and dog telomerase comprising of sequence SEQ ID NO:3, preferably an amino acid consensus sequence of the antigen telomerase comprising of sequence SEQ ID NO:1.

In some embodiments, a kit for the prevention and treatment of telomerase-expressing tumours, the kit comprising a) a pharmaceutical composition, and b) a device for in vivo gene transduction.

In some embodiments, a pharmaceutical composition comprises a nucleotide sequence encoding the amino acid sequence of the antigen telomerase consisting of: an amino acid consensus sequence of the antigen telomerase comprising of sequence SEQ ID NO:1; a chimeric amino acid sequence of dog and human telomerase comprising of sequence SEQ ID NO:2, or a chimeric amino acid sequence of human and dog telomerase comprising of sequence SEQ ID NO:3, preferably an amino acid consensus sequence of the antigen telomerase comprising of sequence SEQ ID NO:1 in combination with one or more excipients and/or adjuvants.

In some embodiments, a protein-based vaccine comprising an amino acid sequence of the antigen telomerase, where the amino acid sequence being selected from the group consisting of: an amino acid consensus sequence of the antigen telomerase comprising of sequence SEQ ID NO:1; a chimeric amino acid sequence of dog and human telomerase comprising of sequence SEQ ID NO: 2, or a chimeric amino acid sequence of human and dog telomerase comprising of sequence SEQ ID NO:3, preferably an amino acid consensus sequence of the antigen telomerase comprising or consisting of sequence SEQ ID NO: 1.

The present invention fits into this context, as it aims to provide a chimeric vaccine capable of increasing the cellular and antibody immune response against telomerase.

According to the present invention, a telomerase consensus between humans and dogs and a nucleotide sequence encoding for said telomerase consensus have now been devised to be advantageously used in the preventive and therapeutic vaccination against telomerase-expressing tumours.

For this purpose, three different approaches have been developed, all of them aimed at creating a chimeric fusion protein from human and dog telomerase. According to the first approach, a chimeric canine-human sequence (CaHu) containing a codon-optimised sequence first of dTERT and then of hTERT has been created, followed by the profilin-like protein of Toxoplasma gondii (PFTG), a protein known for strengthening the immune response [Yarovinsky 2005]. In the second approach, a chimeric human-canine (CaHu) containing a codon-optimised sequence first of hTERT and then of dTERT, followed by PFTG, was created. Finally, in the third approach, a consensus sequence between dTERT and hTERT (conTRT) followed by PFTG was created through a bioinformatic approach.

According to the present invention, it was surprisingly found in a murine model that the genetic vaccine directed against telomerase and encoding for conTRT is more immunogenic and cross-reactive compared to the chimeric sequences CaHu and HuCa. Furthermore, according to the present invention, an antitumour effect was observed in a murine tumour model of colorectal cancer expressing hTert thanks to a preventive treatment with the conTRT vaccine. Both in healthy beagles and in dogs affected by lymphoma, an immunogenicity of the conTRT vaccine against both hTERT and dTERT was observed. The experimental results obtained using a DNA vaccine according to the present invention make it plausible also to assume an anti-tumour effectiveness of administering the protein sequences encoded by said DNA vaccine.

It is therefore a specific object of the present invention an amino acid sequence of the antigen telomerase, said amino acid sequence being selected from among:
an amino acid consensus sequence of the antigen telomerase comprising or consisting of sequence SEQ ID NO:1;
a chimeric amino acid sequence of dog and human telomerase comprising or consisting of sequence SEQ ID NO:2, or
a chimeric amino acid sequence of human and dog telomerase comprising or consisting of sequence SEQ ID NO:3, preferably an amino acid consensus sequence of the antigen telomerase comprising or consisting of sequence SEQ ID NO:1.

The amino acid sequence according to the present invention can further comprise one or more leader sequences such as, for example, the secretion leader sequence of the tissue plasminogen activator (TPA), of IgK, of growth hormone, of serum albumin or of alkaline phosphatase, preferably of the tissue plasminogen activator.

Furthermore, the amino acid sequence according to the present invention can further comprise one or more immunomodulating amino acid sequences, such as, for example, the fragment crystallisable (Fc) region, profilin-like protein of Toxoplasma gondii (PFTG) or a functional fragment derived therefrom, the B subunit of the heat-labile toxin of Escherichia coli (LTB) or the tetanus toxin (TT), preferably profilin-like protein of Toxoplasma gondii.

The present invention further relates to a nucleotide sequence encoding for the amino acid sequence as defined above.

The nucleotide sequence according to the present invention can be selected from a nucleotide sequence comprising or consisting of sequence SEQ ID NO:4, or of a sequence having a sequence identity of at least 80% with respect to SEQ ID NO: 4, of sequence SEQ ID NO:5 or of sequence SEQ ID NO:6, preferably a nucleotide sequence comprising or consisting of sequence SEQ ID NO:4. For example, said sequence having a sequence identity of at least 80% with respect to SEQ ID NO:4 can have a sequence identity of at least 80%, 81%, 82%, 83%, 84%, 85%, 86%, 87%, 88%, 89%, 90%, 91%, 92%, 93%, 94%, 95%, 96%, 97%, 98%, 99%.

As mentioned above, the nucleotide sequence can further comprise a nucleotide sequence encoding for one or more leader sequences such as, for example, the secretion leader sequence of the tissue plasminogen activator (TPA), of IgK, of growth hormone, of serum albumin or of alkaline phosphatase, preferably of the tissue plasminogen activator. The nucleotide sequence can further comprise a nucleotide sequence encoding for one or more immunomodulating amino acid sequences, that is, sequences capable of strengthening the activation of the immune system, such as, for example, the fragment crystallisable (Fc) region, profilin-like protein of Toxoplasma gondii (PFTG) or a functional fragment derived therefrom, the B subunit of the heat-labile toxin of Escherichia coli (LTB) or the tetanus toxin (TT), preferably profilin-like protein of Toxoplasma gondii.

According to a preferred embodiment of the present invention, said nucleotide sequence comprises the nucleotide sequence SEQ ID NO:4, or a sequence having a sequence identity of at least 80% with respect to SEQ ID NO:4, and the nucleotide sequence encoding the profilin-like protein of Toxoplasma gondii. In particular, said nucleotide sequence encoding the profilin-like protein of Toxoplasma gondii can comprise or consist of the sequence TCTAGAAGCGACTGGGACCCCGTGGTGAAGGAATGGCTGGTGGACACCG GCTACTGCTGTGCCGGCGGAATCGCCAACGCCGAGGATGGCGTGGTGTT CGCCGCTGCAGCCGACGATGACGACGGCTGGAGCAAGCTGTACAAGGA CGACCACGAGGAGGACACCATCGGCGAGGACGGCAACGCCTGTGGCAA GGTGTCCATCAACGAGGCCAGCACCATCAAGGCCGCCGTGGACGACGG CAGCGCCCCCAACGGAGTGTGGATCGGCGGCCAGAAATACAAGGTTGTG AGGCCCGAGAAGGGCTTCGAGTACAACGACTGTACCTTCGACATCACCA TGTGTGCCAGAAGCAAAGGCGGAGCCCACCTGATCAAGACCCCCAACGG CAGCATCGTGATCGCCCTGTACGACGAGGAGAAGGAGCAGGACAAGGG GGCTACTGATGA (SEQ ID NO:31), wherein the first six nucleotides of sequence TCTAGA encode for a cloning site of sequence SR, whereas the last six nucleotides of sequence TGATGA encode for two consecutive stop codons. The present invention further relates to an expression vector comprising the nucleotide sequence as defined above.

The expression vector can be selected from the group consisting of a plasmid, for example bacterial plasmids, an RNA, a replicating RNA, amplicons obtained by PCR, a viral vector such as, for example, adenovirus, poxvirus, vaccinia virus, fowlpox, herpes virus, adeno-associated virus (AAV), alphavirus, lentivirus, lambda phage, lymphocytic choriomeningitis virus, Listeria sp, and Salmonella sp.

The present invention further relates to a pharmaceutical composition comprising an amino acid sequence as defined above, a nucleotide sequence as defined above or an expression vector as defined above, in combination with one or more pharmaceutically acceptable excipients and/or adjuvants.

Furthermore, the present invention relates to an amino acid sequence as defined above, a nucleotide sequence as defined above, an expression vector as defined above or a pharmaceutical composition as defined above, for use in the medical field.

It is a further object of the present invention an amino acid sequence as defined above, a nucleotide sequence as defined above, an expression vector as defined above or a pharmaceutical composition as defined above, for use in the prevention and treatment of telomerase-expressing tumours, such as, for example, lymphomas, hemangiosarcoma and prostate cancer.

According to the present invention, the amino acid sequence as defined above, the nucleotide sequence as defined above, the expression vector as defined above or the pharmaceutical composition as defined above can be advantageously employed as a DNA, RNA or protein-based vaccine. When the vaccine is a DNA or RNA vaccine, said vaccine can be administered by electroporation, preferably under the following conditions: 8 pulses of 20 msec, each at 110V, 8 Hz, with an interval of 120 msec between each of them.

According to the present invention, the above-described use can be a use either in the human sector or in the veterinary sector, for example in man or in dogs.

The present invention further relates to a kit for the prevention and treatment of telomerase-expressing tumours, said kit comprising or consisting of: a) nucleotide sequence as defined above, expression vector as defined above or pharmaceutical composition as defined above comprising said nucleotide sequence or said vector, and b) a device for in vivo gene transduction, such as, for example, a device for administration by electroporation.

The present invention will now be described, by an illustrative but non-limiting way, according to a preferred embodiment thereof, with particular reference to the examples and the figures in the appended drawings, wherein:

### BRIEF DESCRIPTION OF THE DRAWINGS

FIG. 1 shows the homology between the amino acid sequences of hTERT, HuCa, dTERT, CaHu and conTRT: A) amino acids 1-500; B) amino acids 501-1000; C) amino acids 1001-1141.

FIG. 2 shows the number of splenocytes (expressed as spot-forming cells, SFC per million) secreting IFN determined by means of the ELISpot technique, after 16 hours of stimulation with pools A, B, C and D of 15-mer peptides and overlapping by 11 amino acid residues both of the hTERT protein and of the dTERT protein, isolated from wild-type mice vaccinated, by electroporation, with the hTERT, dTERT, CaHu, HuCa and conTRT vaccines. The white circles indicate the number of SFC per single mouse, the black circle indicates the geometric mean of SFC of the group.

FIG. 3 shows the tumour volume of a group of Balb/c mice vaccinated with the conTRT vaccine prior to the inoculation of tumour cells (prophylactic setting) compared to the tumour volume measured in the group of unvaccinated control mice.

FIG. 4 shows the number of PBMCs isolated from healthy dogs and secreting IFNγ determined by means of the ELISpot technique, after 16 hours of stimulation with pools A, B, C and D of 15-mer peptides and overlapping by 11 amino acid residues both of the hTERT protein and of the dTERT protein. The graph shows the specific immune response for dog and human telomerase measured in the group of dogs vaccinated with 5 mg of DNA 4 weeks after the last vaccination.

FIG. 5 shows the number of PBMCs isolated from dogs with lymphoma and secreting IFNγ determined by means of the ELISpot technique, after 16 hours of stimulation with pools A, B, C and D of 15-mer peptides and overlapping by 11 amino acid residues both of the hTERT protein and of the dTERT protein. The graph shows the specific immune response for dog and human telomerase measured in the group of dogs vaccinated with 5 mg of DNA 4 weeks after the last vaccination.

### DETAILED DESCRIPTION

### Example 1. Design of the Consensus Sequence of the Antigen Telomerase and of the Nucleotide Sequence Encoding Said Consensus Sequence and In Vivo Study on Effectiveness in the Preventive and Therapeutic Vaccination for Telomerase-Expressing Tumours

Design of the optimised nucleotide sequence of dTERT and hTERT

The optimised cDNA encoding for the hTERT sequence (SEQ ID NO:7) is:

atgccgcgcgctccccgctgccgagccgtgcgctccctgctgcgcagccactaccgcgaggtgct
gccgctggccacgttcgtgcggcgcctggggccccagggctggcggctggtgcagcgcggggacccggc
ggctttccgcgcgctggtggcccagtgcctggtgtgcgtgccctgggacgcacggccgccccccgccgccc
cctccttccgccaggtgtcctgcctgaaggagctggtggcccgagtgctgcagaggctgtgcgagcgcggc
gcgaagaacgtgctggccttcggcttcgcgctgctggacggggcccgcgggggcccccccgaggccttca
ccaccagcgtgcgcagctacctgcccaacacggtgaccgacgcactgcgggggagcggggcgtggggg
ctgctgctgcgccgcgtgggcgacgacgtgctggttcacctgctggcacgctgcgcgctctttgtgctggtggc
tcccagctgcgcctaccaggtgtgcgggccgccgctgtaccagctcggcgctgccactcaggcccggcccc
cgccacacgctagtggaccccgaaggcgtctgggatgcgaacgggcctggaaccatagcgtcagggag
gccggggtccccctgggcctgccagccccgggtgcgaggaggcgcgggggcagtgccagccgaagtct
gccgttgcccaagaggcccaggcgtggcgctgcccctgagccggagcggacgcccgttgggcaggggtc
ctgggcccacccgggcaggacgcgtggaccgagtgaccgtggtttctgtgtggtgtcacctgccagacccg
ccgaagaagccacctctttggagggtgcgctctctggcacgcgccactcccacccatccgtgggccgccag
caccacgcgggccccccatccacatcgcggccaccacgtccctgggacacgccttgtcccccggtgtacg
ccgagaccaagcacttcctctactcctcaggcgacaaggagcagctgcggccctccttcctactcagctctct
gaggcccagcctgactggcgctcggaggctcgtggagaccatctttctgggttccaggccctggatgccagg
gactccccgcaggttgccccgcctgccccagcgctactggcaaatgcggcccctgtttctggagctgcttggg
aaccacgcgcagtgcccctacggggtgctcctcaagacgcactgcccgctgcgagctgcggtcaccccag
cagccggtgtctgtgcccgggagaagccccagggctctgtggcggcccccgaggaggaggacacagac
ccccgtcgcctggtgcagctgctccgccagcacagcagcccctggcaggtgtacggcttcgtgcgggcctg
cctgcgccggctggtgcccccaggcctctggggctccaggcacaacgaacgccgcttcctcaggaacacc
aagaagttcatctccctggggaagcatgccaagctctcgctgcaggagctgacgtggaagatgagcgtgcg
ggactgcgcttggctgcgcaggagcccaggggttggctgtgttccggccgcagagcaccgtctgcgtgagg
agatcctggccaagttcctgcactggctgatgagtgtgtacgtcgtcgagctgctcaggtctttcttttatgtcacg
gagaccacgtttcaaaagaacaggctctttttctaccggaagagtgtctggagcaagttgcaaagcattgga
atcagacagcacttgaagagggtgcagctgcgggagctgtcggaagcagaggtcaggcagcatcggga
agccaggcccgccctgctgacgtccagactccgcttcatccccaagcctgacgggctgcggccgattgtga
acatggactacgtcgtgggagccagaacgttccgcagagaaaagagggccgagcgtctcacctcgaggg
tgaaggcactgttcagcgtgctcaactacgagcgggcgcggcgccccggcctcctgggcgcctctgtgctg
ggcctggacgatatccacagggcctggcgcaccttcgtgctgcgtgtgcgggcccaggacccgccgcctg
agctgtactttgtcaaggtggatgtgacgggcgcgtacgacaccatcccccaggacaggctcacggaggtc
atcgccagcatcatcaaaccccagaacacgtactgcgtgcgtcggtatgccgtggtccagaaggccgccc
atgggcacgtccgcaaggccttcaagagccacgtctctaccttgacagacctccagccgtacatgcgacag
ttcgtggctcacctgcaggagaccagcccgctgagggatgccgtcgtcatcgagcagagctcctccctgaat
gaggccagcagtggcctcttcgacgtcttcctacgcttcatgtgccaccacgccgtgcgcatcaggggcaag
tcctacgtccagtgccaggggatcccgcagggctccatcctctccacgctgctctgcagcctgtgctacggcg
acatggagaacaagctgtttgcggggattcggcgggacgggctgctcctgcgtttggtggatgatttcttgttgg
tgacacctcacctcacccacgcgaaaaccttcctcaggaccctggtccgaggtgtccctgagtatggctgcgt
ggtgaacttgcggaagacagtggtgaacttccctgtagaagacgaggccctgggtggcacggcttttgttca
gatgccggcccacggcctattcccctggtgcggcctgctgctggatacccggaccctggaggtgcagagcg
actactccagctatgcccggacctccatcagagccagtctcaccttcaaccgcggcttcaaggctgggagg
aacatgcgtcgcaaactctttggggtcttgcggctgaagtgtcacagcctgtttctggatttgcaggtgaacagc
ctccagacggtgtgcaccaacatctacaagatcctcctgctgcaggcgtacaggtttcacgcatgtgtgctgc
agctcccatttcatcagcaagtttggaagaaccccacatttttcctgcgcgtcatctctgacacggcctccctctg
ctactccatcctgaaagccaagaacgcagggatgtcgctgggggccaagggcgccgccggccctctgcc
ctccgaggccgtgcagtggctgtgccaccaagcattcctgctcaagctgactcgacaccgtgtcacctacgt
gccactcctggggtcactcaggacagcccagacgcagctgagtcggaagctcccggggacgacgctgac
tgccctggaggccgcagccaacccggcactgccctcagacttcaagaccatcctggactga

The optimised cDNA encoding for the dTERT sequence (SEQ ID NO:8) is:

atggacgccatgaagaggggcctgtgctgcgtgctgctgctgtgcggagccgtgttcgtgagcccc
agcgagatccccagagcccccagatgtagggccgtgagggccctgctgaggggcagatacagagaggt
gctgcccctggccaccttcctgagaaggctgggccctcctggcagactgctggtccgcagaggcgatcctgc
cgcctttagagccctggtggcccagtgcctggtctgtgtgccttggggagccagacctcctcctgccgcccctt
gcttcaggcaggtgtcctgcctgaaagaactggtggccagggtggtgcagagactgtgcgagaggggcgc
cagaaacgtgctggccttcggctttgccctgctggatggcgctagaggcggccctcctgtggccttcaccacc
tccgtgcggagctacctgcccaacaccgtgaccgagaccctgagaggaagcggagcctggggcctgctg
ctgagaagagtgggcgacgacgtgctgacccacctgctggccagatgcgccctgtacctgctggtcgcccc
tagctgtgcctaccaggtctgcggccctcccctgtatgacctgtgcgcccctgcctctctgcctctgcctgcccct
ggactgcctggcctgccagggctgcctggactgggagctggcgctggcgcctctgccgacctgagaccca
ccagacaggcccagaacagcggcgccagaagaagaagaggcagccccggaagcggcgtgcctctgg
ccaagaggcctcggagaagcgtggcctctgagcccgaaagaggcgcccacagaagcttccccagagcc
cagcagcctcctgtgtctgaggcccctgccgtgacacctgccgtggccgcctctcctgctgcttcttgggaggg
cggacctcctggaaccagacccaccacccccgcctggcacccttatcctggccctcagggcgtgcctcacg
atcctgcccaccccgagaccaagcggttcctgtactgcagcggcggcagagagaggctgaggcccagctt
cctgctgtctgccctgcctcctaccctgagcggagcccggaaactggtggagaccatcttcctgggcagcgct
cctcagaagcctggcgccgctcggagaatgcggaggctgcccgccagatactggcggatgcggcccctgt
tccaggaactgctgggcaaccacgccagatgcccctacagggccctgctgaggacccactgccctctgag
ggccatggccgccaaagagggcagcggcaaccaggcccacagaggcgtgggcatctgccccctggaa
agacccgtggccgctccccaggaacagaccgacagcaccaggctggtgcagctgctgagacagcacag
cagcccctggcaggtgtacgccttcctgagggcctgcctgtgttggctggtgcctaccggcctgtggggcagc
aggcacaaccagaggcggtttctgaggaacgtgaagaagttcatcagcctgggcaagcacgccaagctgt
ccctgcaggaactgacctggaagatgaaagtgcgggactgcacctggctgcacggcaatcctggcgcctg
ttgtgtgcctgccgccgagcacaggcggagggaagagatcctggcccggttcctggtgctggtcgatggcc
acatctacgtggtgaagctgctgcggagcttcttctacgtgaccgagaccaccttccagaaaaataggctgtt
cttctaccggaagagcgtgtggagccagctgcagagcatcggcatcaggcagctgttcaacagcgtgcac
ctgagagagctgtccgaggccgaagtgaggcggcacagagaggccagacccgccctgctgaccagca
ggctgagattcctgcccaagcccagcggcctgaggcccatcgtgaacatggactacatcatgggcgccag
gaccttccacagggacaagaaggtgcagcacctgaccagccagctgaaaaccctgttcagcgtgctgaac
tacgagagggccagaaggcctagcctgctgggcgccagcatgctgggcatggacgacatccacagggc
ctggcggaccttcgtgctgaggatcagggcccagaaccctgccccccagctgtacttcgtgaaggtggccat
caccggcgcctacgacgccctgcctcaggacagactggtggaggtgatcgccaacgtgatcaggcccca
ggaaagcacctactgcgtcaggcactacgccgtggtgcagagaaccgccaggggccacgtgaggaagg
ccttcaagaggcacgtgagcaccttcgccgacctgcagccctacatgaggcagttcgtggagaggctgcag
gagaccagcctgctgagggatgccgtggtgatcgagcagagcagcagcctgaacgaggccggcagctc
cctgttccacctgtttctgaggctggtgcacaaccacgtggtgcggatcggcggcaagagctacatccagtgc
cagggcgtgcctcagggcagcatcctgagcaccctgctgtgcagcctgtgctacggcgacatggaaaggc
ggctgttccctggcatcgagcaggacggcgtgctgctgagactggtggacgacttcctgctggtgacccctca
tctgacccaggcccaggccttcctgagaaccctggtgaagggcgtgcccgagtacggctgcagggccaac
ctgcagaaaaccgccgtgaacttccctgtggaggacggcgctctgggatctgctgcccctctgcagctgcct
gcccactgcctgttcccttggtgcggcctgctgctggacaccaggaccctggaagtgagctgcgactacagc
agctacgcccacaccagcatcagggccagcctgaccttcagccagggcgccaagcccggcaggaacat
gcggaggaagctgctggccgtgctgaggctgaagtgctgcgccctgttcctggacctgcaggtcaacggca
tccacaccgtgtacatgaacgtgtacaaaatcttcctgctgcaggcctacaggttccacgcctgcgtgctgca
gctgcccttcaaccagcccgtgaggaagaaccccagcttcttcctgagggtgatcgccgacaccgccagct
gctgctacagcctgctgaaggccagaaatgccggcctgtctctgggagccaagggcgccagcggcctgttt
cctagcgaggccgccagatggctgtgcctgcacgcctttctgctgaagctggcccaccacagcggcaccta
cagatgcctgctgggagccctgcaggccgccaaagcccacctgagcaggcagctgcctagaggaacact
ggccgccctggaagccgccgctgaccctagcctgaccgccgacttcaagaccatcctggac

### Design of the Optimised Nucleotide Sequence of CaHu and HuCa.

The design of the optimised cDNA encoding for the chimeric CaHu sequence was created on the basis of the dTERT and hTERT sequences, both submitted to the NCBI database with accession numbers NM_001031630.1 and NM_198253.3, respectively.

The optimised cDNA encoding for the CaHu sequence (SEQ ID NO:5) is:

atgggctggtcctgtattattctgtttctggtcgccaccgctaccggagtccatagtcctagagcaccccgctgtc
gcgccgtgagggccctgctgagaggcaggtaccgcgaggtgctgccactggctacctttctgcggagactg
ggaccacctggcaggctgctggtgaggcgaggcgaccctgcagctttccgcgccctggtggctcagtgcct
ggtgtgcgtgccttggggagcaaggccaccacctgcagcaccatgctttcgacaggtgagctgtctgaagg
agctggtcgcacgagtggtccagcgactgtgcgaaaggggcgctcgcaacgtgctggcattcggctttgcc
ctgctggatggagctcgaggaggaccaccagtggccttcaccaccagcgtgcggagctacctgcccaata
ctgtgaccgagacactgaggggatccggagcatggggactgctgctgcgacgagtgggggacgatgtcct
gacacacctgctggcacgctgcgccctgtatctgctggtggctccctcatgcgcataccaggtctgtggccctc
cactgtatgacctgtgcgcacctgccagcctgcccctgcctgccccagggctgcctggactgccaggactgc
caggactgggagctggagcaggggcctcagctgatctgcgacctacccggcaggctcagaacagcgga
gcaagaaggcgccgaggaagtccaggatcaggagtgcctctggcaaagaggccacggagaagcgtcg
catccgagccagaacgaggagctcaccggagcttccctagggcacagcagccacctgtgagtgaggcac
ctgcagtgactccagcagtcgctgcaagtcctgcagcttcatgggaaggaggaccaccaggaacccgacc
tactaccccagcttggcatccataccctggaccacagggagtgccacacgaccctgcccatccagagacc
aagcggtttctgtattgcagcgggggacgagaacggctgagaccaagcttcctgctgtccgccctgcctcca
acactgagtggggctagaaaactggtggagactatctttctgggatcagctccacagaagcctggagcagc
aaggcgaatgcgacggctgcctgccaggtactggaggatgcgcccactgttccaggagctgctgggaaac
cacgctcgatgcccctatcgagcactgctgcggacacattgtcctctgcgggcaatggctgcaaaggaagg
gagtggaaatcaggcacaccgaggagtgggaatctgccccctggagagacctgtcgcagctccacagga
acagaccgacagcacacgactggtgcagctgctgcgccagcatagctccccatggcaggtgtacgcctttc
tgagagcttgcctgtgctggctggtgccaaccggactgtgggggtccaggcacaaccagagaaggtttctgc
gcaatgtgaagaaattcatctccctgggcaagcatgccaaactgtctctgcaggagctgacctggaagatga
aagtgagggactgtacatggctgcacggaaacccaggagcttgctgcgtgcctgcagcagaacatcgccg
acgggaggaaatcctggccagatttctggtgctggtcgatggacacatctacgtggtcaaactgctgaggtct
ttcttttatgtgaccgagacaactttccagaagaataggctgttcttttatcgcaagagcgtgtggagtaaactgc
agtctatcggcattagacagcacctgaaaagagtgcagctgagggagctgagtgaggccgaagtcagac
agcatagggaagctcgccctgcactgctgacaagccgactgcggttcatccccaagcctgacgggctgcg
cccaattgtgaacatggattacgtggtcggagcacggacctttagaagggagaaacgagccgaacggctg
acatcaagagtgaaggctctgttcagcgtcctgaattatgagagggcacgccgacccggactgctgggagc
ctctgtgctggggctggacgacatccacagagcttggaggacctttgtgctgagagtcagggcacaggacc
cccctccagagctgtacttcgtgaaggtcgcaatcaccggagcctatgacacaattccacaggatcgcctga
ctgaagtgattgccagcatcatcaagccccagaatacctactgcgtgcggagatatgcagtggtccagaag
gctgcacacggccatgtgcggaaggcctttaaatcacacgtcagcactctgaccgatctgcagccttacatg
cgccagttcgtggctcatctgcaggagacttctccactgcgggacgcagtggtcatcgagcagtctagttcact
gaacgaagctagctccgggctgttcgacgtgttcctgaggttcatgtgccaccatgccgtgcgcattcgagga
aaatcctacgtccagtgtcagggaatcccacagggctccattctgtctaccctgctgtgctctctgtgctatggc
gacatggagaataagctgtttgcaggcatcaggcgagatggactgctgctgagactggtggacgattttctgc
tggtcaccccccacctgacacatgccaaaactttcctgcgcaccctggtgcgaggagtccctgaatacggct
gcgtggtcaacctgaggaagacagtggtcaatttcccagtggaggacgaagccctgggaggaactgctttt
gtccagatgccagcacacggactgttcccatggtgtggactgctgctggacacacgcactctggaggtgca
gagcgattactctagttatgcccggacatctatcagagctagtctgacttttaaccgggggttcaaggccggaa
gaaatatgcgacggaaactgtttggcgtgctgcggctgaagtgccatagtctgttcctggacctgcaggtgaa
ctcactgcagactgtctgtaccaatatctacaaaattctgctgctgcaggcatatagatttcacgcctgcgtgct
gcagctgccattccatcagcaggtctggaagaaccccacttttttctgagagtgatcagcgataccgctagc
ctgtgctactccattctgaaggccaaaaatgctggaatgtccctgggagcaaaaggagcagctggaccact
gccatctgaggctgtgcagtggctgtgccaccaggcattcctgctgaagctgactcggcatagagtgacctat
gtcccactgctgggaagcctgcggacagcccagactcagctgtccagaaagctgccaggaaccacactg
accgccctggaagcagccgctaacccagctctgcccagcgactttaaaacaatcctggat

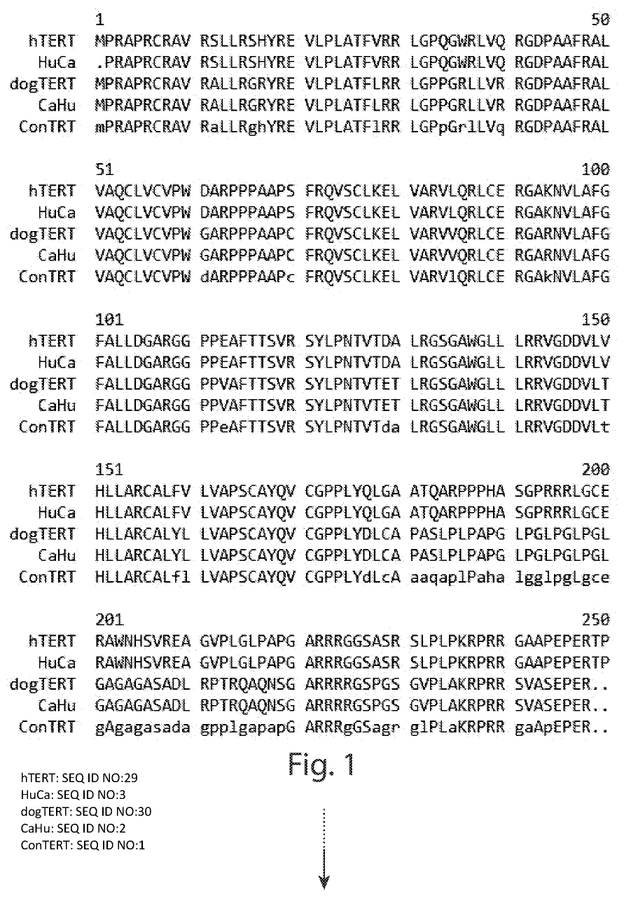

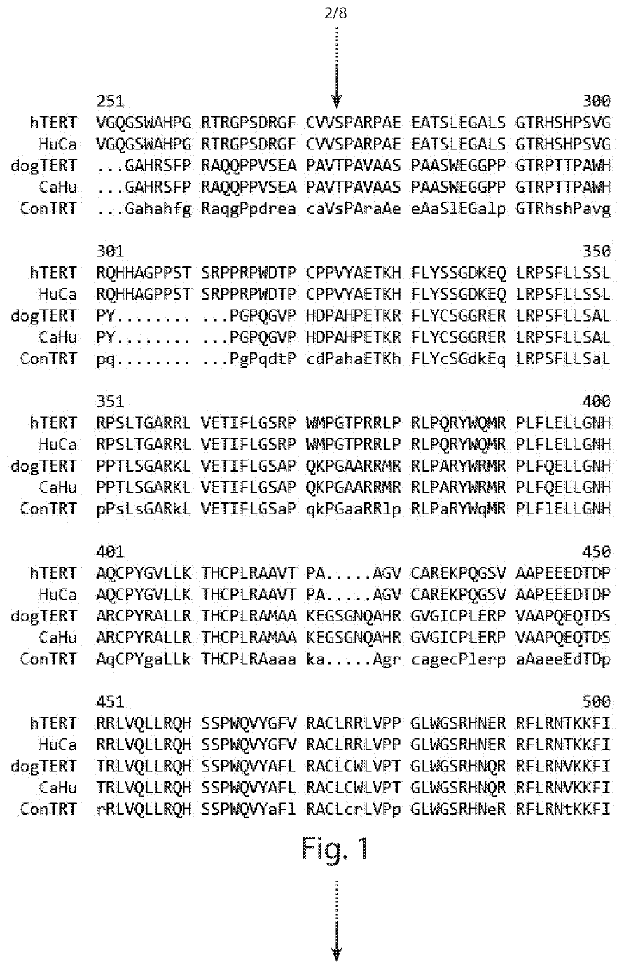

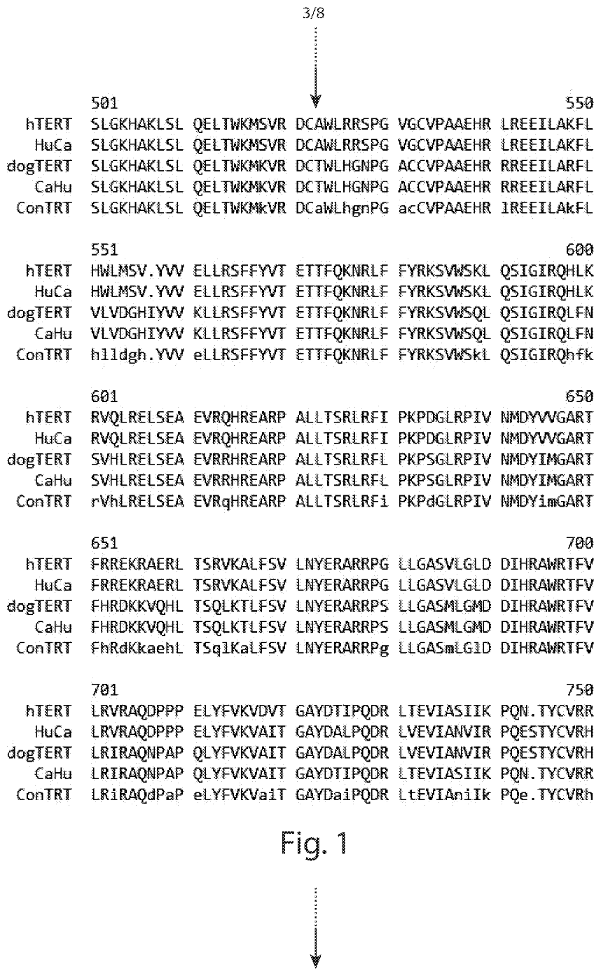

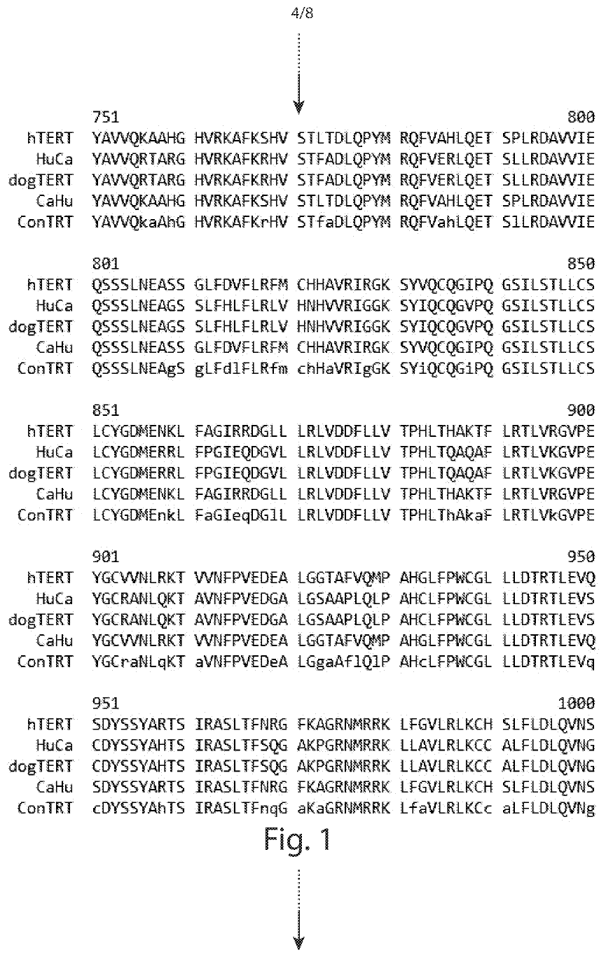

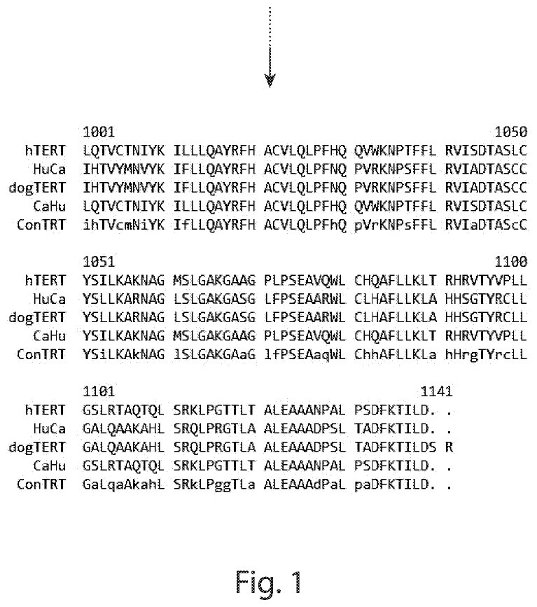

The following CaHu amino acid sequence (SEQ ID NO:2) was obtained from the fusion (FIGS. 1-3):

MPRAPRCRAVRALLRGRYREVLPLATFLRRLGPPGRLLVRRGDPAAFRA
LVAQCLVCVPWGARPPPAAPCFRQVSCLKELVARVVQRLCERGARNVLA
FGFALLDGARGGPPVAFTTSVRSYLPNTVTETLRGSGAWGLLLRRVGDD
VLTHLLARCALYLLVAPSCAYQVCGPPLYDLCAPASLPLPAPGLPGLPG
LPGLGAGAGASADLRPTRQAQNSGARRRRGSPGSGVPLAKRPRRSVASE
PERGAHRSFPRAQQPPVSEAPAVTPAVAASPAASWEGGPPGTRPTTPAW
HPYPGPQGVPHDPAHPETKRFLYCSGGRERLRPSFLLSALPPTLSGARK
LVETIFLGSAPQKPGAARRMRRLPARYWRMRPLFQELLGNHARCPYRAL
LRTHCPLRAMAAKEGSGNQAHRGVGICPLERPVAAPQEQTDSTRLVQLL
RQHSSPWQVYAFLRACLCWLVPTGLWGSRHNQRRFLRNVKKFISLGKHA
KLSLQELTWKMKVRDCTWLHGNPGACCVPAAEHRRREEILARFLVLVDG
HIYVVKLLRSFFYVTETTFQKNRLFFYRKSVWSQLQSIGIRQLFNSVHL
RELSEAEVRRHREARPALLTSRLRFLPKPSGLRPIVNMDYIMGARTFHR
DKKVQHLTSQLKTLFSVLNYERARRPSLLGASMLGMDDIHRAWRTFVLR
IRAQNPAPQLYFVKVAITGAYDTIPQDRLTEVIASIIKPQNTYCVRRYA
VVQKAAHGHVRKAFKSHVSTLTDLQPYMRQFVAHLQETSPLRDAVVIEQ
SSSLNEASSGLFDVFLRFMCHHAVRIRGKSYVQCQGIPQGSILSTLLCS
LCYGDMENKLFAGIRRDGLLLRLVDDFLLVTPHLTHAKTFLRTLVRGVP
EYGCVVNLRKTVVNFPVEDEALGGTAFVQMPAHGLFPWCGLLLDTRTLE
VQSDYSSYARTSIRASLTFNRGFKAGRNMRRKLFGVLRLKCHSLFLDLQ
VNSLQTVCTNIYKILLLQAYRFHACVLQLPFHQQVWKNPTFFLRVISDT
ASLCYSILKAKNAGMSLGAKGAAGPLPSEAVQWLCHQAFLLKLTRHRVT
YVPLLGSLRTAQTQLSRKLPGTTLTALEAAANPALPSDFKTILD

The design of the optimised cDNA encoding for the chimeric HuCa sequence was created on the basis of the hTERT and dTERT sequences, both submitted to the NCBI database with accession numbers NM_198253.3 and NM_001031630.1, respectively.

The optimised cDNA encoding for the sequence HuCa (SEQ ID NO:6) is:

atgggatggtcttgtattattctgttcctggtcgccactgccaccggggtccacagccctagagcacctagatgt
agagccgtgagaagtctgctgcgctcacactaccgagaggtgctgcctctggccacattcgtccggagactg
ggaccacagggatggcgactggtgcagagaggcgatccagcagcttttagagctctggtcgcacagtgcct
ggtgtgcgtgccatgggacgcacgaccacctccagcagcccctagcttccggcaggtgtcctgcctgaaag
aactggtggcaagggtcctgcagcggctgtgcgagcgaggagctaagaacgtgctggcattcggatttgca
ctgctggatggagcacgaggaggaccacctgaggcctttaccacaagcgtgcggtcctatctgcccaatac
agtcactgacgctctgagaggcagcggagcatggggactgctgctgaggcgagtgggcgacgatgtgctg
gtccacctgctggcacgatgcgctctgttcgtgctggtcgctccttcctgcgcataccaggtgtgcggaccacc
actgtatcagctgggagctgcaacccaggcaagacctccaccacacgctagtggacctcgacggagactg
ggatgtgaaagggcttggaaccattcagtgcgcgaggcaggagtcccactgggactgccagcacctggag
caaggcgccgaggaggaagtgcctcacgaagcctgccactgccaaagcgaccacgaagaggagcag
ctcctgaaccagagaggactcccgtgggacagggatcctgggcacacccaggaaggacccgeggaccc
tcagatagaggcttctgcgtggtcagccctgctaggccagcagaggaagccactagtctggagggcgccct
gtcagggaccagacactctcatcccagtgtgggcaggcagcaccatgctgggcctccatccacatctcggc
cccctagaccatgggatactccctgtccacccgtgtacgccgaaaccaaacatttcctgtatagctccggcga
caaggagcagctgcgcccaagttttctgctgtctagtctgcgaccatcactgaccggagcaaggcgcctggt
ggaaacaatcttcctgggaagcaggccctggatgcctggaactccacgacggctgccacgactgcctcag
agatactggcagatgcgccctctgtttctggagctgctgggaaaccacgcacagtgcccatatggagtgctg
ctgaaaacacattgtcccctgagggcagcagtgactcctgctgcaggcgtctgcgcacgagagaagccac
agggaagcgtggcagctccagaggaagaggacaccgatcctagaaggctggtgcagctgctgaggcag
cactcaagcccttggcaggtgtacggattcgtccgcgcatgtctgcgccgactggtgcctccaggactgtggg
gaagccgccacaacgaacggagattcctgcgaaataccaagaagttcatctccctggggaagcatgcca
aactgtctctgcaggagctgacatggaaaatgtcagtgagggactgcgcttggctgaggcgcagccctgga
gtgggatgcgtgccagcagcagagcaccgactgcgagaagagattctggccaagttcctgcattggctgat
gagcgtgtacgtggtcgaactgctgcgctccttcttttatgtcaccgagactacctttcagaagaacagactgtt
cttttataggaaatcagtgtggagccagctgcagagcatcggcattagacagctgttcaatagcgtgcacctg
agggaactgtccgaagcagaggtccgacggcatagggaggctcgaccagcactgctgaccagccggct
gaggtttctgcccaaacctagtggactgaggcccatcgtgaacatggattacattatgggcgccaggactttc
caccgcgacaagaaagtgcagcatctgacctctcagctgaagacactgtttagtgtgctgaattatgagcga
gcaagaaggccctctctgctgggagctagtatgctggggatggacgacatccaccgagcatggcggacctt
cgtgctgcgcattcgagcccagaacccagctccccagctgtactttgtgaaggtcgccatcacaggagccta
tgacgctctgccacaggataggctggtggaagtcatcgccaatgtgattcgaccacaggagtccacctactg
cgtccggcattatgcagtggtccagagaacagccaggggccacgtgcgcaaggctttcaaacgacacgtg
agcaccttcgccgacctgcagccatacatgcggcagtttgtggaaagactgcaggagaccagcctgctgcg
agacgcagtggtcattgaacagtcctctagtctgaacgaggctggctcaagcctgttccacctgtttctgcgcct
ggtgcacaatcatgtggtccggatcgggggaaagagttacattcagtgtcagggagtgccccagggctcca
tcctgtctaccctgctgtgctccctgtgctatggcgatatggaacgccgactgttccccggaattgagcaggac
ggcgtgctgctgcgactggtggacgatttcctgctggtgactcctcatctgacccaggcccaggcttttctgcgg
acactggtgaaaggggtccccgaatacggatgcagagctaacctgcagaagactgcagtgaatttccctgt
cgaggacggggccctgggatctgctgcacctctgcagctgccagctcactgcctgtttccatggtgtggcctg
ctgctggatacccggacactggaggtgagctgtgactactcctcttatgcccatacaagcatcagagcttccct
gactttctctcagggggccaagcccggaagaaacatgcggagaaaactgctggcagtgctgaggctgaa
gtgctgtgccctgtttctggatctgcaggtgaacggcatccacaccgtgtacatgaatgtctataaaattttcctg
ctgcaggcataccggtttcatgcctgcgtgctgcagctgcccttcaaccagcctgtcagaaagaatcctagctt
ctttctgagagtgatcgcagacacagccagttgctgttattcactgctgaaagctagaaatgcaggactgtccc
tgggagcaaagggagcttcaggactgttcccaagcgaagccgetaggtggctgtgcctgcacgcatttctgc
tgaaactggcccaccatagcggaacttaccgatgtctgctgggcgctctgcaggcagccaaggcacatctgt
cccgacagctgccacgagggaccctggctgcactggaggcagctgcagacccttctctgactgccgatttc
aaaaccatcctggac

The following HuCa amino acid sequence (SEQ ID NO:3) was obtained from the fusion (FIGS. 1-3):

PRAPRCRAVRSLLRSHYREVLPLATFVRRLGPQGWRLVQRGDPAAFRAL
VAQCLVCVPWDARPPPAAPSFRQVSCLKELVARVLQRLCERGAKNVLAF
GFALLDGARGGPPEAFTTSVRSYLPNTVTDALRGSGAWGLLLRRVGDDV
LVHLLARCALFVLVAPSCAYQVCGPPLYQLGAATQARPPPHASGPRRRL
GCERAWNHSVREAGVPLGLPAPGARRRGGSASRSLPLPKRPRRGAAPEP
ERTPVGQGSWAHPGRTRGPSDRGFCVVSPARPAEEATSLEGALSGTRHS
HPSVGRQHHAGPPSTSRPPRPWDTPCPPVYAETKHFLYSSGDKEQLRPS
FLLSSLRPSLTGARRLVETIFLGSRPWMPGTPRRLPRLPQRYWQMRPLF
LELLGNHAQCPYGVLLKTHCPLRAAVTPAAGVCAREKPQGSVAAPEEED
TDPRRLVQLLRQHSSPWQVYGFVRACLRRLVPPGLWGSRHNERRFLRNT
KKFISLGKHAKLSLQELTWKMSVRDCAWLRRSPGVGCVPAAEHRLREEI
LAKFLHWLMSVYVVELLRSFFYVTETTFQKNRLFFYRKSVWSKLQSIGI
RQHLKRVQLRELSEAEVRQHREARPALLTSRLRFIPKPDGLRPIVNMDY
VVGARTFRREKRAERLTSRVKALFSVLNYERARRPGLLGASVLGLDDIH
RAWRTFVLRVRAQDPPPELYFVKVAITGAYDALPQDRLVEVIANVIRPQ
ESTYCVRHYAVVQRTARGHVRKAFKRHVSTFADLQPYMRQFVERLQETS
LLRDAVVIEQSSSLNEAGSSLFHLFLRLVHNHVVRIGGKSYIQCQGVPQ
GSILSTLLCSLCYGDMERRLFPGIEQDGVLLRLVDDFLLVTPHLTQAQA
FLRTLVKGVPEYGCRANLQKTAVNFPVEDGALGSAAPLQLPAHCLFPWC
GLLLDTRTLEVSCDYSSYAHTSIRASLTFSQGAKPGRNMRRKLLAVLRL
KCCALFLDLQVNGIHTVYMNVYKIFLLQAYRFHACVLQLPFNQPVRKNP
SFFLRVIADTASCCYSLLKARNAGLSLGAKGASGLFPSEAARWLCLHAF
LLKLAHHSGTYRCLLGALQAAKAHLSRQLPRGTLAALEAAADPSLTADF
KTILD

The cDNA encoding for these sequences was optimised for codon usage. The cDNA optimisation consists in replacing the original codons with nucleotide triplets recognised by the tRNAs which are most frequent and efficient in the cells of the organism of interest. For this purpose use was made of an algorithm (GeneOptimizer, Thermofisher) that allows the cDNA of interest to be designed so as to increase the levels of expression in cells of the species in which it is desired to use the antigen (Homo sapiens). Furthermore, any undesired restriction sites or splicing sites generated in silico by this operation were removed. The DNA was synthesised by Invitrogen GeneArt (Germany) and cloned in the pTK1A-TPA vector.

Design of the conTRT Consensus Sequence.

The design of the optimised cDNA encoding for the consensus sequence conTRT was created on the basis of the dTERT and hTERT sequences by means of a bioinformatic method, by overlapping the sequences of the dog and human epitopes differing by a single amino acid and optimised for binding with human HLAs.

Table 1 shows a list of the dTERT and hTERT epitopes differing by a single amino acid and optimised for human HLAs.

TABLE 1

| | | | Improved |
|---|---|---|---|
| | Starting | | HLA Allele |
| Species | Position | Epitope | Binding |
| Human | 4 | APRCRAVRS (SEQ ID NO: 9) | B7 |
| Dog | 4 | APRCRAVRA (SEQ ID NO: 10) | B7 |
| Human | 20 | EVLPLATFV (SEQ ID NO: 11) | B7 |
| Dog | 20 | EVLPLATFL (SEQ ID NO: 12) | B7 |
| Human | 554 | VELLRSFFY (SEQ ID NO: 13) | B*1503 |
| Dog | 544 | VKLLRSFFY (SEQ ID NO: 14) | B*1503 |
| Human | 616 | LLTSRLRFI (SEQ ID NO: 15) | A2 |
| Dog | 606 | LLTSRLRFL (SEQ ID NO: 16) | A2 |
| Human | 631 | RPIVNMDYV (SEQ ID NO: 17) | B51, B53 |
| Dog | 621 | RPIVNMDYI (SEQ ID NO: 18) | B51, B53 |
| Human | 659 | KALFSVLNY (SEQ ID NO: 19) | B58 |
| Dog | 649 | KTLFSVLNY (SEQ ID NO: 20) | B58 |
| Human | 682 | GLDDIHRAW (SEQ ID NO: 21) | A32 |
| Dog | 672 | GMDDIHRAW (SEQ ID NO: 22) | A32 |
| Human | 689 | AWRTFVLRV (SEQ ID NO: 23) | A24 |
| dog | 679 | AWRTFVLRI (SEQ ID NO: 24) | A24 |
| Human | 784 | SPLRDAVVI (SEQ ID NO: 25) | A68 |
| Dog | 775 | SLLRDAVVI (SEQ ID NO: 26) | A68 |

The optimised cDNA encoding for the conTRT sequence (SEQ ID NO:4) is:

atgggatggtcatgtattattctgttcctggtcgctaccgcaaccggagtgcatagtccaagagcccc
tagatgtcgagccgtgagggcactgctgcgcagccgataccgggaggtgctgcctctggctaccttcctgcg
gagactgggaccacagggatggagactggtgaggcgaggggacccagcagcttttagggctctggtcgc
acagtgcctggtgtgcgtgccatggggagcaagaccacctccagcagccccttcattcaggcaggtgagct
gcctgaaagagctggtggctagggtcctgcagcggctgtgcgaacgaggagcaaagaacgtgctggcttt
cgggtttgcactgctggacggagctagaggaggaccacctgtggccttcaccaccagcgtgcggagctatc
tgcccaataccgtgacagatactctgcgaggatccggagcatggggactgctgctgcgacgggggggga
cgatgtgctggtccacctgctggcacgatgcgctctgttcctgctggtggccccttcttgcgcttaccaggtctgt
ggaccacccctgtatcagctgggcgccccaacaagtctgagacctccaccccctgcttcaccaccaagaa
ggcgcctgccaggactgagggcatggaaccatgccgtgcgcgacctgcgagtcactcgacagctgcaga
atagcggagcacgacggagaaggggatccccaagctcctctctgccactgcctaagcgaccacgccgat
ctgtggcaagtgagcctgaacgaaccccagtcggacgaggagcttggagatcccctccaagaacaaggc
agccatctgtgagtggcttcccagtggtctctccagcagtcccagcaagccctgctacctcctgggagggag
caccatccggaacaagaccatctactccagcatggggaaggcagcaccatgctggacccccttcaacaa
gcagatacccaaggccatggggagtgcctcacccaccagtccatccagagactaaacggttcctgtatagtt
caggaggcaaggaacgcctgcgaccctcttttctgctgagtgcactgcgaccttccctgtctggagcacgaa
agctggtggagactatcttcctgggaagccgcccttggatgccaggaaccccacggagactgaggcgcct
gcctcagcggtactggcgaatgagaccactgtttcaggaactgctgggaaaccacgccaggtgcccatatc
gcgtgctgctgaaaacacattgtccactgcgggcaatggtgactcccgaggcctccgtcaatcagagacac
aagggagtgggaatttgcccacagggaagcgtggtcgcacctccacaggaacagacagactccactcgc
ctggtgcagctgctgcgacagcatagctccccctggcaggtgtacgcttttctgcgagcatgtctgcggtggct
ggtgcctacaggactgtggggaagccgccacaaccagcgacggttcctgcggaacgtgaagaagttcatc
tctctgggcaagcatgccaaactgagtctgcaggagctgacctggaagatgtccgtgcgcgattgcacatgg
ctgagaaggtctccaggagtgggatgcgtgcctgctgcagaacaccgccgacgggaggaaattctggcca
aattcctggtgtggctgatgagtcatatctacgtggtcaagctgctgcggtcattcttttatgtgaccgagactacc
tttcagaagaaccgactgttcttttatcggaaatcagtgtggagccagctgcagtccatcggcattcgccagct
gctgaacagcgtgcagctgcgagagctgagtgaggcagaagtcagaaggcaccgcgaagcacgacctg
ccctgctgacttcaaggctgcgcttcatccctaaaccaagcggcctgaggccaattgtgaacatggactacat
catgggggctcgcaccttccgccgagataagaaagtgcagagactgacctcaaggctgaagacactgttta
gcgtgctgaattatgagagagctcggagacctagtctgctgggagcatcaatgctgggcctggacgatattc
accgggcatggagaaccttcgtgctgcgaatccgggcacagaacccacctccacagctgtactttgtgaag
gtcgccattactggcgcttatgacaccatcccccaggataggctggtggaggtcatcgcctccatcatcaagc
ctcaggaatctacatactgcgtgaggcgctatgctgtggtccagaagactgcacgcgggcacgtgcgaaag
gctttcaaatcccatgtctctaccctgacagacctgcagccatacatgagacagtttgtggagaggctgcagg
aaacaagccccctgcgcgatgcagtggtcattgagcagtctagttcactgaacgaagctagctcctctctgttc
cacctgtttctgcggctgatgcacaatcatgtggtcagaatcaggggcaaatcttacatccagtgtcaggggat
tccccaaggaagtatcctgtcaaccctgctgtgcagcctgtgctatggggacatggagcgcaagctgttcccc
gggatccgacgggatggactgctgctgcggctggtggacgatttcctgctggtcacccctcacctgacacag
gcccagacttttctgagaaccctggtgaaaggcgtcccagagtacgggtgcgtggtcaacctgaggaagac
tgtggtcaatttccccgtggaagacggggctctgggatccaccgcaccactgcagctgcctgcacatggact
gtttccttggtgtggactgctgctggacactagaaccctggaggtgagttcagattacagctcctatgcccgga
cttcaattagagctagcctgaccttctccagaggctttaagccagggaggaacatgagaaggaaactgctg
gccgtgctgaggctgaagtgccacgctctgtttctggacctgcaggtgaacagcatccagaccgtctacaca
aatatctataaaattctgctgctgcaggcctacagattccatgcttgcgtgctgcagctgcccttcaaccagcag
gtctggaagaatccctccttctttctgagagtgatcgctgataccgcatctctgtgctatagtatcctgaaggcca
aaaatgctggactgtctctgggagcaaaaggagcagctggaccactgcctagtgaggcagtgcggtggct
gtgcctgcaggccttcctgctgaagctgacaagacacagcgtgacttacgtcccactgctgggcgcactgag
gactgcccagacccagctgtccaggcagctgcctcgcacaactctgacagccctggaagcagccgctaac
ccagcactgaccgccgacttcaaaacaattctggat

The new conTRT amino acid consensus sequence (SEQ ID NO:1) (FIGS. 1-3) is:

MPRAPRCRAVRALLRSRYREVLPLATFLRRLGPQGWRLVRRGDPAA
FRALVAQCLVCVPWGARPPPAAPSFRQVSCLKELVARVLQRLCERGAKN
VLAFGFALLDGARGGPPVAFTTSVRSYLPNTVTDTLRGSGAWGLLLRRV
GDDVLVHLLARCALFLLVAPSCAYQVCGPPLYQLGAPTSLRPPPPASPP
RRRLPGLRAWNHAVRDLRVTRQLQNSGARRRRGSPSSSLPLPKRPRRSV
ASEPERTPVGRGAWRSPPRTRQPSVSGFPVVSPAVPASPATSWEGAPSG
TRPSTPAWGRQHHAGPPSTSRYPRPWGVPHPPVHPETKRFLYSSGGKER
LRPSFLLSALRPSLSGARKLVETIFLGSRPWMPGTPRRLRRLPQRYWRM
RPLFQELLGNHARCPYRVLLKTHCPLRAMVTPEASVNQRHKGVGICPQG
SVVAPPQEQTDSTRLVQLLRQHSSPWQVYAFLRACLRWLVPTGLWGSRH
NQRRFLRNVKKFISLGKHAKLSLQELTWKMSVRDCTWLRRSPGVGCVPA
AEHRRREEILAKFLVWLMSHIYVVKLLRSFFYVTETTFQKNRLFFYRKS
VWSQLQSIGIRQLLNSVQLRELSEAEVRRHREARPALLTSRLRFIPKPS
GLRPIVNMDYIMGARTFRRDKKVQRLTSRLKTLFSVLNYERARRPSLLG
ASMLGLDDIHRAWRTFVLRIRAQNPPPQLYFVKVAITGAYDTIPQDRLV
EVIASIIKPQESTYCVRRYAVVQKTARGHVRKAFKSHVSTLTDLQPYMR
QFVERLQETSPLRDAVVIEQSSSLNEASSSLFHLFLRLMHNHVVRIRGK
SYIQCQGIPQGSILSTLLCSLCYGDMERKLFPGIRRDGLLLRLVDDFLL
VTPHLTQAQTFLRTLVKGVPEYGCVVNLRKTVVNFPVEDGALGSTAPLQ
LPAHGLFPWCGLLLDTRTLEVSSDYSSYARTSIRASLTFSRGFKPGRNM
RRKLLAVLRLKCHALFLDLQVNSIQTVYTNIYKILLLQAYRFHACVLQL
PFNQQVWKNPSFFLRVIADTASLCYSILKAKNAGLSLGAKGAAGPLPSE
AVRWLCLQAFLLKLTRHSVTYVPLLGALRTAQTQLSRQLPRTTLTALEA
AANPALTADFKTILD

Evaluation of the Immunogenicity of the CaHu, HuCa and conTRT Sequences.

For the purpose of evaluating the cell-mediated immune response induced by the CaHu, HuCa and conTRT constructs, a genetic vaccination approach based on electroporation into the skeletal muscle (DNA-EP) of wild-type mice was adopted. This vaccination technique enables the use of laboratory-engineered constructs of different sizes and does not induce a neutralising response as in the case of viral vectors, thus making it possible to repeat the vaccinations a number of times. The immunisation protocol consisted in 4 injections, into the quadriceps of Balb/c mice, of 50 μg of the pTK1A-tPA-hTERT-LTB, pTK1A-tPA-dTERT-LTB, pTK1A-CaHu-PFTG, pTK1A-HuCa-PFTG and pTK1A-conTRT-PFTG constructs, spaced apart from one another by 1 week. In detail, the vector used is the pTK1A vector. The pTK1A expression vector comprises the promoter and intron A from human cytomegalovirus (CMV), a polylinker site for the cloning and bovine growth hormone (bGH) as a polyA for the transcription termination. Furthermore, the first two constructs pTK1A-tPA-hTERT-LTB and pTK1A-tPA-dTERT-LTB contain, in addition to the gene for human and dog telomerase, the leader sequence of tissue plasminogen activator (tPA), a signal sequence that favours the secretion of the protein of interest, and the sequence of lymphotoxin beta (LTB), which increases the immune response to the vector. The leader sequence of tissue plasminogen activator (TPA) is ATGGATGCAATGAAGAGAGGGCTCTGCTGTGTGCTGCTGCTGTGTGGAG CAGTCTTCGTTTCGCCCAGC (SEQ ID NO:27), which encodes for the amino acid sequence MDAMKRGLCCVLLLCGAVFVSPS (SEQ ID NO:28).

The other three constructs pTK1A-CaHu-PFTG, pTK1A-HuCa-PFTG and pTK1A-conTRT-PFTG comprise, in addition to the chimeric sequences CaHu and HuCA or the TRT consensus sequence, the PFTG sequence, which increases the immune response to the vector.

The DNA was formulated in phosphate-buffered saline (PBS) at a concentration of 1 mg/ml. DNA-EP was performed with a BTX 830 electroporator and flat electrodes with a distance of 0.5 cm (BTX, Harward apparatus), under the following electric conditions in the Low Voltage mode: 2 pulses of 60 msec at 100V, 250 msec pause between pulses. In order to measure the immune response induced by the vaccine, the number of splenocytes (expressed as spot-forming cells, SFC per million) secreting IFN was determined by means of the ELISpot technique, after 16 hours of stimulation with pools A, B, C and D of 15-mer peptides and overlapping by 11 amino acid residues both of the hTERT protein and of the dTERT protein. In particular, pool A comprises the amino acids 1-296; pool B comprises the amino acids 297-587; pool C comprises the amino acids 588-867; and pool D comprises the amino acids 868-1139. The splenocytes were isolated from wild-type mice vaccinated, by electroporation, with the hTERT, dTERT, CaHu, HuCa and conTRT vaccines.

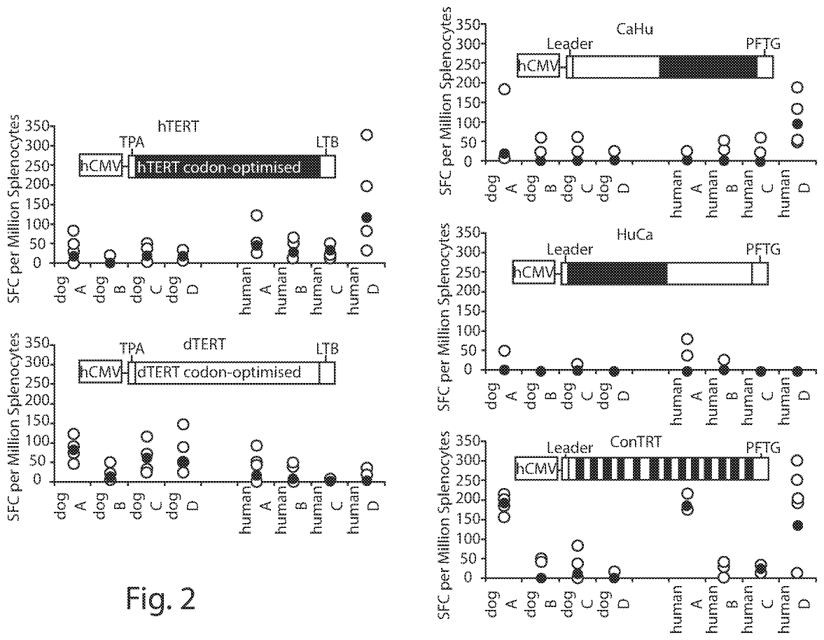

As may be seen from FIG. 2, the mice immunised with pTK1-hTERT responded to the vaccine (mean response of the group greater than 50 SFC) when stimulated with the pools of hTERT peptides (above all pools A and D), whilst the response to the pools of dTERT peptides was very low or absent. In contrast, the mice immunised with pTK1-dTERT responded to the vaccine after stimulation with the dTERT pools (in particular pools A, C and D), but not with the hTERT pools. As regards the mice immunised with the pTK1-CaHu construct, a substantial response was observed only after stimulation with pool D of hTERT, whereas in the mice immunised with the pTK1-HuCa construct, no response towards any pool of peptides was detected. Finally, splenocytes isolated from mice immunised with the pTK1-conTRT-PFTG construct showed a substantial response after stimulation both with pool A of hTERT peptides and with pools A and D of dTERT peptides. These results indicate that the construct with the conTRT consensus sequence is strongly immunogenic and capable of arousing new specificities against the antigen telomerase, of both the human type and canine type.

Evaluation of the Antitumour Effect of the conTRT Sequence in Mice with Tumours.

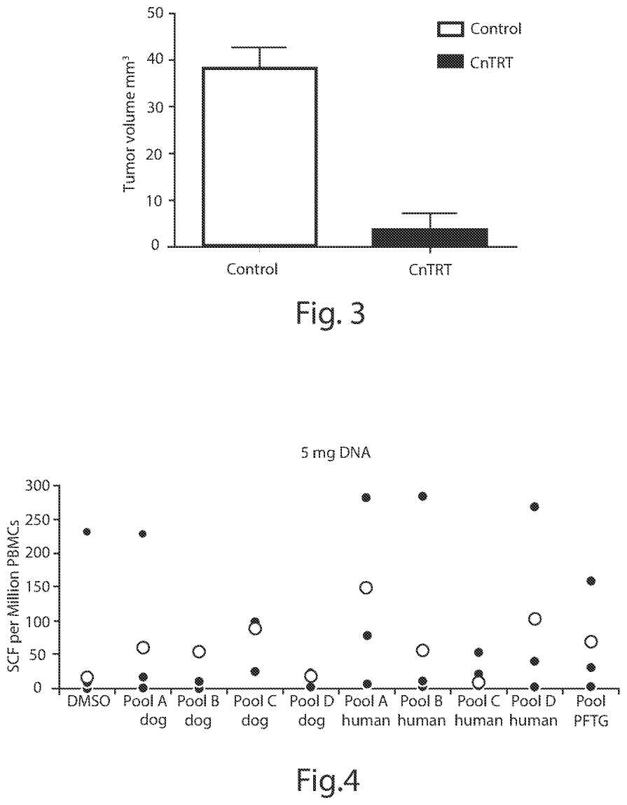

For the purpose of evaluating the antitumour effect of the conTRT vaccine, Balb/c mice received three doses of the conTRT vaccine at two week intervals following a prophylactic vaccination protocol and were then injected with tumour cells of colon carcinoma (CT26) expressing human telomerase. As shown by FIG. 3, the vaccinated mice demonstrated less tumour growth compared to unvaccinated mice.

Evaluation of the Immunogenicity of the conTRT Sequence in Healthy Dogs and Dogs with Lymphoma.

For the purpose of determining the minimum immunogenic dose of the DNA produced, a clinical study was conducted on 9 healthy dogs of the beagle breed (6 males and 3 females reared and treated at Meditox cro-Czech Republic) vaccinated with a single intramuscular injection followed by electroporation with tapered doses of DNA, in particular 5 mg, 1 mg and 0.3 mg of plasmid DNA. The animals received three doses of vaccine every two weeks and peripheral blood samples were taken before every vaccination and at the end of the study (one month after the last dose of vaccine) in order to isolate the PBMCs (peripheral blood mononuclear cells). With the aim of identifying the minimal dose of DNA capable of inducing the maximum immune response specific for the consensus telomerase, the PBMCs were then analysed for the induction of an immune response by means of the ELISPOT assay, the immunological assay most widely used to evaluate vaccines in clinical studies in view of its high sensitivity. Briefly, the PBMCs were stimulated with different pools of immunogenic peptides, of both dog and human telomerase, and the production of IFN□ was then measured in order to evaluate the T-cell mediated immune response specific for telomerase. Prior to the start of the clinical study, at time zero, the possible presence of a response against telomerase was measured by ELISPOT and none was detected in any of the dogs treated. After two doses of DNA, in the group of dogs vaccinated with the maximum dose of 5 mg of DNA, the immune response specific for dog telomerase (measured after stimulation of the PBMCs with the pools of dog telomerase peptides, A, B, C and D) and for human telomerase (measured after stimulation of the PBMCs with the pools of human telomerase peptides, A, B, C and D) was still very low, if not absent. In the groups vaccinated with 1 and 0.3 mg doses, no telomerase-specific response was detected. In contrast, as shown in FIG. 4, 4 weeks after the third and last vaccination dose, in the group of dogs vaccinated with the maximum dose of DNA (5 mg), a substantial response was measured, both towards the pools of dog telomerase and towards the pools of human telomerase, thus confirming the choice of that vaccination scheme as optimal for obtaining, after a 4-week period, a greater specific response, and demonstrating, in particular, the effectiveness of the genetic vaccine pTK1A-conTRT-PFTG in inducing a telomerase-specific immune response. Furthermore, a considerable immune response specific for the PFTG protein was also measured in the same group. In the group of dogs vaccinated with an intermediate dose (1 mg), the measured response was considerably lower, whilst no response was detected in the group of dogs vaccinated with the minimum dose (0.3 mg), thus suggesting that 5 mg represents the optimal dose for inducing an efficient immune response.

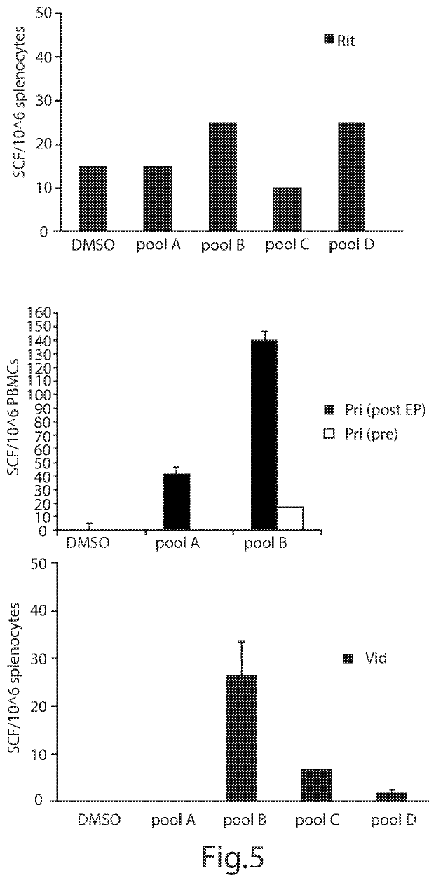

The same vaccination protocol was used in three dogs affected by lymphoma expressing dog telomerase (FIG. 5). Four weeks after the last vaccination PBMCs were isolated from peripheral blood and the telomerase-specific immune response was measured by measuring the cells secreting IFN, after stimulation with the pools of dogTERT peptides. In particular, a substantial telomerase-specific response towards pool B of dogTERT was measured.

1. An amino acid sequence of the antigen telomerase, wherein the amino acid sequence being selected from the group consisting of:
an amino acid consensus sequence of the antigen telomerase comprising sequence SEQ ID NO:1;
a chimeric amino acid sequence of dog and human telomerase comprising sequence SEQ ID NO:2, or
a chimeric amino acid sequence of human and dog telomerase comprising sequence SEQ ID NO:3, preferably an amino acid consensus sequence of the antigen telomerase comprising sequence SEQ ID NO:1.

2. The amino acid sequence according to claim 1, wherein the amino acid sequence further comprises one or more leader sequences.

3. The amino acid sequence according to claim 1, wherein the amino acid sequence further comprises one or more immunomodulating amino acid sequences.

4. A nucleotide sequence encoding the amino acid sequence of claim 1.

5. The nucleotide sequence according to claim 4, wherein the nucleotide sequence is selected from the group consisting of a nucleotide sequence comprising sequence SEQ ID NO:4, or a nucleotide sequence comprising a sequence having a sequence identity of at least 80% with respect to SEQ ID NO: 4, a nucleotide sequence comprising SEQ ID NO:5 or a nucleotide sequence comprising SEQ ID NO:6.

6. A nucleotide sequence encoding the amino acid sequence of claim 2.

7. A nucleotide sequence encoding the amino acid sequence of claim 3.

8. The nucleotide sequence according to claim 5, wherein the nucleotide sequence comprises the nucleotide sequence SEQ ID NO: 4, or a sequence having a sequence identity of at least 80% with respect to SEQ ID NO:4, and a nucleotide sequence encoding the profilin-like protein of Toxoplasma gondii.

9. The nucleotide sequence according to claim 8, wherein the nucleotide sequence encoding the profilin-like protein of Toxoplasma gondii comprises SEQ ID NO: 31.

10. An expression vector comprising the nucleotide sequence as defined in claim 4.

11. An expression vector according to claim 10, wherein said vector is selected from the group consisting of a plasmid, an RNA, a replicating RNA, amplicons obtained by PCR, and a viral vector.

12. A pharmaceutical composition comprising an amino acid sequence as defined in claim 1, in combination with one or more excipients and/or adjuvants.

13. (canceled)

14. A method of vaccinating a subject against telomerase-expressing tumors or treating a subject having a telomerase-expressing tumors comprising administering an amino acid sequence according to claim 1 to the subject.

15. A DNA, or RNA based vaccine comprising the nucleotide sequence of claim 4.

16. A method of vaccinating a subject against telomerase-expressing tumors or treating a subject having a telomerase-expressing tumors, comprising administering the vaccine of claim 15 to the subject by electroporation.

17. (canceled)

18. A kit for the prevention and treatment of telomerase-expressing tumours, the kit comprising a) a pharmaceutical composition as defined in claim 12, and b) a device for in vivo gene transduction.

19. The amino acid sequence according to claim 2, wherein the one or more leader sequence is a secretion leader sequence of a protein selected from the group consisting of tissue plasminogen activator (TPA), IgK, growth hormone, serum albumin, and alkaline phosphatase.

20. The amino acid sequence according to claim 3, wherein the one or more immunomodulating amino acid sequences are selected from the group consisting of the fragment crystallisable (Fc) region, profilin-like protein of Toxoplasma gondii (PFTG) or a functional fragment derived therefrom, the B subunit of the heat-labile toxin of Escherichia coli (LTB) and the tetanus toxin (TT).

21. A pharmaceutical composition comprising a nucleotide sequence as defined in claim 4, in combination with one or more excipients and/or adjuvants.

22. A protein-based vaccine comprising the amino acid sequence of claim 1.


SEQUENCE LISTING (31 sequences)

SEQ ID NO: 1 - protein, 1139 aa, Artificial
  consensus sequence of telomerase antigen
MPRAPRCRAVRALLRSRYREVLPLATFLRRLGPQGWRLVRRGDPAAFRALVAQCLVCVPWGARPPPAAPSFRQVSCLKELVARVLQRLCERGAKNVLAFGFALLDGARGGPPVAFTTSVRSYLPNTVTDTLRGSGAWGLLLRRVGDDVLVHLLARCALFLLVAPSCAYQVCGPPLYQLGAPTSLRPPPPASPPRRRLPGLRAWNHAVRDLRVTRQLQNSGARRRRGSPSSSLPLPKRPRRSVASEPERTPVGRGAWRSPPRTRQPSVSGFPVVSPAVPASPATSWEGAPSGTRPSTPAWGRQHHAGPPSTSRYPRPWGVPHPPVHPETKRFLYSSGGKERLRPSFLLSALRPSLSGARKLVETIFLGSRPWMPGTPRRLRRLPQRYWRMRPLFQELLGNHARCPYRVLLKTHCPLRAMVTPEASVNQRHKGVGICPQGSVVAPPQEQTDSTRLVQLLRQHSSPWQVYAFLRACLRWLVPTGLWGSRHNQRRFLRNVKKFISLGKHAKLSLQELTWKMSVRDCTWLRRSPGVGCVPAAEHRRREEILAKFLVWLMSHIYVVKLLRSFFYVTETTFQKNRLFFYRKSVWSQLQSIGIRQLLNSVQLRELSEAEVRRHREARPALLTSRLRFIPKPSGLRPIVNMDYIMGARTFRRDKKVQRLTSRLKTLFSVLNYERARRPSLLGASMLGLDDIHRAWRTFVLRIRAQNPPPQLYFVKVAITGAYDTIPQDRLVEVIASIIKPQESTYCVRRYAVVQKTARGHVRKAFKSHVSTLTDLQPYMRQFVERLQETSPLRDAVVIEQSSSLNEASSSLFHLFLRLMHNHVVRIRGKSYIQCQGIPQGSILSTLLCSLCYGDMERKLFPGIRRDGLLLRLVDDFLLVTPHLTQAQTFLRTLVKGVPEYGCVVNLRKTVVNFPVEDGALGSTAPLQLPAHGLFPWCGLLLDTRTLEVSSDYSSYARTSIRASLTFSRGFKPGRNMRRKLLAVLRLKCHALFLDLQVNSIQTVYTNIYKILLLQAYRFHACVLQLPFNQQVWKNPSFFLRVIADTASLCYSILKAKNAGLSLGAKGAAGPLPSEAVRWLCLQAFLLKLTRHSVTYVPLLGALRTAQTQLSRQLPRTTLTALEAAANPALTADFKTILD

SEQ ID NO: 2 - protein, 1122 aa, Artificial
  chimeric fusion protein of dog and human telomerase
MPRAPRCRAVRALLRGRYREVLPLATFLRRLGPPGRLLVRRGDPAAFRALVAQCLVCVPWGARPPPAAPCFRQVSCLKELVARVVQRLCERGARNVLAFGFALLDGARGGPPVAFTTSVRSYLPNTVTETLRGSGAWGLLLRRVGDDVLTHLLARCALYLLVAPSCAYQVCGPPLYDLCAPASLPLPAPGLPGLPGLPGLGAGAGASADLRPTRQAQNSGARRRRGSPGSGVPLAKRPRRSVASEPERGAHRSFPRAQQPPVSEAPAVTPAVAASPAASWEGGPPGTRPTTPAWHPYPGPQGVPHDPAHPETKRFLYCSGGRERLRPSFLLSALPPTLSGARKLVETIFLGSAPQKPGAARRMRRLPARYWRMRPLFQELLGNHARCPYRALLRTHCPLRAMAAKEGSGNQAHRGVGICPLERPVAAPQEQTDSTRLVQLLRQHSSPWQVYAFLRACLCWLVPTGLWGSRHNQRRFLRNVKKFISLGKHAKLSLQELTWKMKVRDCTWLHGNPGACCVPAAEHRRREEILARFLVLVDGHIYVVKLLRSFFYVTETTFQKNRLFFYRKSVWSQLQSIGIRQLFNSVHLRELSEAEVRRHREARPALLTSRLRFLPKPSGLRPIVNMDYIMGARTFHRDKKVQHLTSQLKTLFSVLNYERARRPSLLGASMLGMDDIHRAWRTFVLRIRAQNPAPQLYFVKVAITGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 3 - protein, 1132 aa, Artificial
  chimeric fusion protein of human and dog telomerase
PRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVAITGAYDALPQDRLVEVIANVIRPQESTYCVRHYAVVQRTARGHVRKAFKRHVSTFADLQPYMRQFVERLQETSLLRDAVVIEQSSSLNEAGSSLFHLFLRLVHNHVVRIGGKSYIQCQGVPQGSILSTLLCSLCYGDMERRLFPGIEQDGVLLRLVDDFLLVTPHLTQAQAFLRTLVKGVPEYGCRANLQKTAVNFPVEDGALGSAAPLQLPAHCLFPWCGLLLDTRTLEVSCDYSSYAHTSIRASLTFSQGAKPGRNMRRKLLAVLRLKCCALFLDLQVNGIHTVYMNVYKIFLLQAYRFHACVLQLPFNQPVRKNPSFFLRVIADTASCCYSLLKARNAGLSLGAKGASGLFPSEAARWLCLHAFLLKLAHHSGTYRCLLGALQAAKAHLSRQLPRGTLAALEAAADPSLTADFKTILD

SEQ ID NO: 4 - DNA, 3471 bp, Artificial
  nucleotide sequence encoding SEQ ID NO1
atgggatggtcatgtattattctgttcctggtcgctaccgcaaccggagtgcatagtccaagagcccctagatgtcgagccgtgagggcactgctgcgcagccgataccgggaggtgctgcctctggctaccttcctgcggagactgggaccacagggatggagactggtgaggcgaggggacccagcagcttttagggctctggtcgcacagtgcctggtgtgcgtgccatggggagcaagaccacctccagcagccccttcattcaggcaggtgagctgcctgaaagagctggtggctagggtcctgcagcggctgtgcgaacgaggagcaaagaacgtgctggctttcgggtttgcactgctggacggagctagaggaggaccacctgtggccttcaccaccagcgtgcggagctatctgcccaataccgtgacagatactctgcgaggatccggagcatggggactgctgctgcgacgggtgggggacgatgtgctggtccacctgctggcacgatgcgctctgttcctgctggtggccccttcttgcgcttaccaggtctgtggaccacccctgtatcagctgggcgccccaacaagtctgagacctccaccccctgcttcaccaccaagaaggcgcctgccaggactgagggcatggaaccatgccgtgcgcgacctgcgagtcactcgacagctgcagaatagcggagcacgacggagaaggggatccccaagctcctctctgccactgcctaagcgaccacgccgatctgtggcaagtgagcctgaacgaaccccagtcggacgaggagcttggagatcccctccaagaacaaggcagccatctgtgagtggcttcccagtggtctctccagcagtcccagcaagccctgctacctcctgggagggagcaccatccggaacaagaccatctactccagcatggggaaggcagcaccatgctggacccccttcaacaagcagatacccaaggccatggggagtgcctcacccaccagtccatccagagactaaacggttcctgtatagttcaggaggcaaggaacgcctgcgaccctcttttctgctgagtgcactgcgaccttccctgtctggagcacgaaagctggtggagactatcttcctgggaagccgcccttggatgccaggaaccccacggagactgaggcgcctgcctcagcggtactggcgaatgagaccactgtttcaggaactgctgggaaaccacgccaggtgcccatatcgcgtgctgctgaaaacacattgtccactgcgggcaatggtgactcccgaggcctccgtcaatcagagacacaagggagtgggaatttgcccacagggaagcgtggtcgcacctccacaggaacagacagactccactcgcctggtgcagctgctgcgacagcatagctccccctggcaggtgtacgcttttctgcgagcatgtctgcggtggctggtgcctacaggactgtggggaagccgccacaaccagcgacggttcctgcggaacgtgaagaagttcatctctctgggcaagcatgccaaactgagtctgcaggagctgacctggaagatgtccgtgcgcgattgcacatggctgagaaggtctccaggagtgggatgcgtgcctgctgcagaacaccgccgacgggaggaaattctggccaaattcctggtgtggctgatgagtcatatctacgtggtcaagctgctgcggtcattcttttatgtgaccgagactacctttcagaagaaccgactgttcttttatcggaaatcagtgtggagccagctgcagtccatcggcattcgccagctgctgaacagcgtgcagctgcgagagctgagtgaggcagaagtcagaaggcaccgcgaagcacgacctgccctgctgacttcaaggctgcgcttcatccctaaaccaagcggcctgaggccaattgtgaacatggactacatcatgggggctcgcaccttccgccgagataagaaagtgcagagactgacctcaaggctgaagacactgtttagcgtgctgaattatgagagagctcggagacctagtctgctgggagcatcaatgctgggcctggacgatattcaccgggcatggagaaccttcgtgctgcgaatccgggcacagaacccacctccacagctgtactttgtgaaggtcgccattactggcgcttatgacaccatcccccaggataggctggtggaggtcatcgcctccatcatcaagcctcaggaatctacatactgcgtgaggcgctatgctgtggtccagaagactgcacgcgggcacgtgcgaaaggctttcaaatcccatgtctctaccctgacagacctgcagccatacatgagacagtttgtggagaggctgcaggaaacaagccccctgcgcgatgcagtggtcattgagcagtctagttcactgaacgaagctagctcctctctgttccacctgtttctgcggctgatgcacaatcatgtggtcagaatcaggggcaaatcttacatccagtgtcaggggattccccaaggaagtatcctgtcaaccctgctgtgcagcctgtgctatggggacatggagcgcaagctgttccccgggatccgacgggatggactgctgctgcggctggtggacgatttcctgctggtcacccctcacctgacacaggcccagacttttctgagaaccctggtgaaaggcgtcccagagtacgggtgcgtggtcaacctgaggaagactgtggtcaatttccccgtggaagacggggctctgggatccaccgcaccactgcagctgcctgcacatggactgtttccttggtgtggactgctgctggacactagaaccctggaggtgagttcagattacagctcctatgcccggacttcaattagagctagcctgaccttctccagaggctttaagccagggaggaacatgagaaggaaactgctggccgtgctgaggctgaagtgccacgctctgtttctggacctgcaggtgaacagcatccagaccgtctacacaaatatctataaaattctgctgctgcaggcctacagattccatgcttgcgtgctgcagctgcccttcaaccagcaggtctggaagaatccctccttctttctgagagtgatcgctgataccgcatctctgtgctatagtatcctgaaggccaaaaatgctggactgtctctgggagcaaaaggagcagctggaccactgcctagtgaggcagtgcggtggctgtgcctgcaggccttcctgctgaagctgacaagacacagcgtgacttacgtcccactgctgggcgcactgaggactgcccagacccagctgtccaggcagctgcctcgcacaactctgacagccctggaagcagccgctaacccagcactgaccgccgacttcaaaacaattctggat

SEQ ID NO: 5 - DNA, 3420 bp, Artificial
  nucleotide sequence encoding SEQ ID NO2
atgggctggtcctgtattattctgtttctggtcgccaccgctaccggagtccatagtcctagagcaccccgctgtcgcgccgtgagggccctgctgagaggcaggtaccgcgaggtgctgccactggctacctttctgcggagactgggaccacctggcaggctgctggtgaggcgaggcgaccctgcagctttccgcgccctggtggctcagtgcctggtgtgcgtgccttggggagcaaggccaccacctgcagcaccatgctttcgacaggtgagctgtctgaaggagctggtcgcacgagtggtccagcgactgtgcgaaaggggcgctcgcaacgtgctggcattcggctttgccctgctggatggagctcgaggaggaccaccagtggccttcaccaccagcgtgcggagctacctgcccaatactgtgaccgagacactgaggggatccggagcatggggactgctgctgcgacgagtgggggacgatgtcctgacacacctgctggcacgctgcgccctgtatctgctggtggctccctcatgcgcataccaggtctgtggccctccactgtatgacctgtgcgcacctgccagcctgcccctgcctgccccagggctgcctggactgccaggactgccaggactgggagctggagcaggggcctcagctgatctgcgacctacccggcaggctcagaacagcggagcaagaaggcgccgaggaagtccaggatcaggagtgcctctggcaaagaggccacggagaagcgtcgcatccgagccagaacgaggagctcaccggagcttccctagggcacagcagccacctgtgagtgaggcacctgcagtgactccagcagtcgctgcaagtcctgcagcttcatgggaaggaggaccaccaggaacccgacctactaccccagcttggcatccataccctggaccacagggagtgccacacgaccctgcccatccagagaccaagcggtttctgtattgcagcgggggacgagaacggctgagaccaagcttcctgctgtccgccctgcctccaacactgagtggggctagaaaactggtggagactatctttctgggatcagctccacagaagcctggagcagcaaggcgaatgcgacggctgcctgccaggtactggaggatgcgcccactgttccaggagctgctgggaaaccacgctcgatgcccctatcgagcactgctgcggacacattgtcctctgcgggcaatggctgcaaaggaagggagtggaaatcaggcacaccgaggagtgggaatctgccccctggagagacctgtcgcagctccacaggaacagaccgacagcacacgactggtgcagctgctgcgccagcatagctccccatggcaggtgtacgcctttctgagagcttgcctgtgctggctggtgccaaccggactgtgggggtccaggcacaaccagagaaggtttctgcgcaatgtgaagaaattcatctccctgggcaagcatgccaaactgtctctgcaggagctgacctggaagatgaaagtgagggactgtacatggctgcacggaaacccaggagcttgctgcgtgcctgcagcagaacatcgccgacgggaggaaatcctggccagatttctggtgctggtcgatggacacatctacgtggtcaaactgctgaggtctttcttttatgtgaccgagacaactttccagaagaataggctgttcttttatcgcaagagcgtgtggagtaaactgcagtctatcggcattagacagcacctgaaaagagtgcagctgagggagctgagtgaggccgaagtcagacagcatagggaagctcgccctgcactgctgacaagccgactgcggttcatccccaagcctgacgggctgcgcccaattgtgaacatggattacgtggtcggagcacggacctttagaagggagaaacgagccgaacggctgacatcaagagtgaaggctctgttcagcgtcctgaattatgagagggcacgccgacccggactgctgggagcctctgtgctggggctggacgacatccacagagcttggaggacctttgtgctgagagtcagggcacaggacccccctccagagctgtacttcgtgaaggtcgcaatcaccggagcctatgacacaattccacaggatcgcctgactgaagtgattgccagcatcatcaagccccagaatacctactgcgtgcggagatatgcagtggtccagaaggctgcacacggccatgtgcggaaggcctttaaatcacacgtcagcactctgaccgatctgcagccttacatgcgccagttcgtggctcatctgcaggagacttctccactgcgggacgcagtggtcatcgagcagtctagttcactgaacgaagctagctccgggctgttcgacgtgttcctgaggttcatgtgccaccatgccgtgcgcattcgaggaaaatcctacgtccagtgtcagggaatcccacagggctccattctgtctaccctgctgtgctctctgtgctatggcgacatggagaataagctgtttgcaggcatcaggcgagatggactgctgctgagactggtggacgattttctgctggtcaccccccacctgacacatgccaaaactttcctgcgcaccctggtgcgaggagtccctgaatacggctgcgtggtcaacctgaggaagacagtggtcaatttcccagtggaggacgaagccctgggaggaactgcttttgtccagatgccagcacacggactgttcccatggtgtggactgctgctggacacacgcactctggaggtgcagagcgattactctagttatgcccggacatctatcagagctagtctgacttttaaccgggggttcaaggccggaagaaatatgcgacggaaactgtttggcgtgctgcggctgaagtgccatagtctgttcctggacctgcaggtgaactcactgcagactgtctgtaccaatatctacaaaattctgctgctgcaggcatatagatttcacgcctgcgtgctgcagctgccattccatcagcaggtctggaagaaccccactttctttctgagagtgatcagcgataccgctagcctgtgctactccattctgaaggccaaaaatgctggaatgtccctgggagcaaaaggagcagctggaccactgccatctgaggctgtgcagtggctgtgccaccaggcattcctgctgaagctgactcggcatagagtgacctatgtcccactgctgggaagcctgcggacagcccagactcagctgtccagaaagctgccaggaaccacactgaccgccctggaagcagccgctaacccagctctgcccagcgactttaaaacaatcctggat

SEQ ID NO: 6 - DNA, 3453 bp, Artificial
  nucleotide sequence encoding SEQ ID NO3
atgggatggtcttgtattattctgttcctggtcgccactgccaccggggtccacagccctagagcacctagatgtagagccgtgagaagtctgctgcgctcacactaccgagaggtgctgcctctggccacattcgtccggagactgggaccacagggatggcgactggtgcagagaggcgatccagcagcttttagagctctggtcgcacagtgcctggtgtgcgtgccatgggacgcacgaccacctccagcagcccctagcttccggcaggtgtcctgcctgaaagaactggtggcaagggtcctgcagcggctgtgcgagcgaggagctaagaacgtgctggcattcggatttgcactgctggatggagcacgaggaggaccacctgaggcctttaccacaagcgtgcggtcctatctgcccaatacagtcactgacgctctgagaggcagcggagcatggggactgctgctgaggcgagtgggcgacgatgtgctggtccacctgctggcacgatgcgctctgttcgtgctggtcgctccttcctgcgcataccaggtgtgcggaccaccactgtatcagctgggagctgcaacccaggcaagacctccaccacacgctagtggacctcgacggagactgggatgtgaaagggcttggaaccattcagtgcgcgaggcaggagtcccactgggactgccagcacctggagcaaggcgccgaggaggaagtgcctcacgaagcctgccactgccaaagcgaccacgaagaggagcagctcctgaaccagagaggactcccgtgggacagggatcctgggcacacccaggaaggacccgcggaccctcagatagaggcttctgcgtggtcagccctgctaggccagcagaggaagccactagtctggagggcgccctgtcagggaccagacactctcatcccagtgtgggcaggcagcaccatgctgggcctccatccacatctcggccccctagaccatgggatactccctgtccacccgtgtacgccgaaaccaaacatttcctgtatagctccggcgacaaggagcagctgcgcccaagttttctgctgtctagtctgcgaccatcactgaccggagcaaggcgcctggtggaaacaatcttcctgggaagcaggccctggatgcctggaactccacgacggctgccacgactgcctcagagatactggcagatgcgccctctgtttctggagctgctgggaaaccacgcacagtgcccatatggagtgctgctgaaaacacattgtcccctgagggcagcagtgactcctgctgcaggcgtctgcgcacgagagaagccacagggaagcgtggcagctccagaggaagaggacaccgatcctagaaggctggtgcagctgctgaggcagcactcaagcccttggcaggtgtacggattcgtccgcgcatgtctgcgccgactggtgcctccaggactgtggggaagccgccacaacgaacggagattcctgcgaaataccaagaagttcatctccctggggaagcatgccaaactgtctctgcaggagctgacatggaaaatgtcagtgagggactgcgcttggctgaggcgcagccctggagtgggatgcgtgccagcagcagagcaccgactgcgagaagagattctggccaagttcctgcattggctgatgagcgtgtacgtggtcgaactgctgcgctccttcttttatgtcaccgagactacctttcagaagaacagactgttcttttataggaaatcagtgtggagccagctgcagagcatcggcattagacagctgttcaatagcgtgcacctgagggaactgtccgaagcagaggtccgacggcatagggaggctcgaccagcactgctgaccagccggctgaggtttctgcccaaacctagtggactgaggcccatcgtgaacatggattacattatgggcgccaggactttccaccgcgacaagaaagtgcagcatctgacctctcagctgaagacactgtttagtgtgctgaattatgagcgagcaagaaggccctctctgctgggagctagtatgctggggatggacgacatccaccgagcatggcggaccttcgtgctgcgcattcgagcccagaacccagctccccagctgtactttgtgaaggtcgccatcacaggagcctatgacgctctgccacaggataggctggtggaagtcatcgccaatgtgattcgaccacaggagtccacctactgcgtccggcattatgcagtggtccagagaacagccaggggccacgtgcgcaaggctttcaaacgacacgtgagcaccttcgccgacctgcagccatacatgcggcagtttgtggaaagactgcaggagaccagcctgctgcgagacgcagtggtcattgaacagtcctctagtctgaacgaggctggctcaagcctgttccacctgtttctgcgcctggtgcacaatcatgtggtccggatcgggggaaagagttacattcagtgtcagggagtgccccagggctccatcctgtctaccctgctgtgctccctgtgctatggcgatatggaacgccgactgttccccggaattgagcaggacggcgtgctgctgcgactggtggacgatttcctgctggtgactcctcatctgacccaggcccaggcttttctgcggacactggtgaaaggggtccccgaatacggatgcagagctaacctgcagaagactgcagtgaatttccctgtcgaggacggggccctgggatctgctgcacctctgcagctgccagctcactgcctgtttccatggtgtggcctgctgctggatacccggacactggaggtgagctgtgactactcctcttatgcccatacaagcatcagagcttccctgactttctctcagggggccaagcccggaagaaacatgcggagaaaactgctggcagtgctgaggctgaagtgctgtgccctgtttctggatctgcaggtgaacggcatccacaccgtgtacatgaatgtctataaaattttcctgctgcaggcataccggtttcatgcctgcgtgctgcagctgcccttcaaccagcctgtcagaaagaatcctagcttctttctgagagtgatcgcagacacagccagttgctgttattcactgctgaaagctagaaatgcaggactgtccctgggagcaaagggagcttcaggactgttcccaagcgaagccgctaggtggctgtgcctgcacgcatttctgctgaaactggcccaccatagcggaacttaccgatgtctgctgggcgctctgcaggcagccaaggcacatctgtcccgacagctgccacgagggaccctggctgcactggaggcagctgcagacccttctctgactgccgatttcaaaaccatcctggac

SEQ ID NO: 7 - DNA, 3399 bp, Artificial
  nucleotide sequence encoding SEQ ID NO29
atgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactga

SEQ ID NO: 8 - DNA, 3441 bp, Artificial
  nucleotide sequence encoding SEQ ID NO30
atggacgccatgaagaggggcctgtgctgcgtgctgctgctgtgcggagccgtgttcgtgagccccagcgagatccccagagcccccagatgtagggccgtgagggccctgctgaggggcagatacagagaggtgctgcccctggccaccttcctgagaaggctgggccctcctggcagactgctggtccgcagaggcgatcctgccgcctttagagccctggtggcccagtgcctggtctgtgtgccttggggagccagacctcctcctgccgccccttgcttcaggcaggtgtcctgcctgaaagaactggtggccagggtggtgcagagactgtgcgagaggggcgccagaaacgtgctggccttcggctttgccctgctggatggcgctagaggcggccctcctgtggccttcaccacctccgtgcggagctacctgcccaacaccgtgaccgagaccctgagaggaagcggagcctggggcctgctgctgagaagagtgggcgacgacgtgctgacccacctgctggccagatgcgccctgtacctgctggtcgcccctagctgtgcctaccaggtctgcggccctcccctgtatgacctgtgcgcccctgcctctctgcctctgcctgcccctggactgcctggcctgccagggctgcctggactgggagctggcgctggcgcctctgccgacctgagacccaccagacaggcccagaacagcggcgccagaagaagaagaggcagccccggaagcggcgtgcctctggccaagaggcctcggagaagcgtggcctctgagcccgaaagaggcgcccacagaagcttccccagagcccagcagcctcctgtgtctgaggcccctgccgtgacacctgccgtggccgcctctcctgctgcttcttgggagggcggacctcctggaaccagacccaccacccccgcctggcacccttatcctggccctcagggcgtgcctcacgatcctgcccaccccgagaccaagcggttcctgtactgcagcggcggcagagagaggctgaggcccagcttcctgctgtctgccctgcctcctaccctgagcggagcccggaaactggtggagaccatcttcctgggcagcgctcctcagaagcctggcgccgctcggagaatgcggaggctgcccgccagatactggcggatgcggcccctgttccaggaactgctgggcaaccacgccagatgcccctacagggccctgctgaggacccactgccctctgagggccatggccgccaaagagggcagcggcaaccaggcccacagaggcgtgggcatctgccccctggaaagacccgtggccgctccccaggaacagaccgacagcaccaggctggtgcagctgctgagacagcacagcagcccctggcaggtgtacgccttcctgagggcctgcctgtgttggctggtgcctaccggcctgtggggcagcaggcacaaccagaggcggtttctgaggaacgtgaagaagttcatcagcctgggcaagcacgccaagctgtccctgcaggaactgacctggaagatgaaagtgcgggactgcacctggctgcacggcaatcctggcgcctgttgtgtgcctgccgccgagcacaggcggagggaagagatcctggcccggttcctggtgctggtcgatggccacatctacgtggtgaagctgctgcggagcttcttctacgtgaccgagaccaccttccagaaaaataggctgttcttctaccggaagagcgtgtggagccagctgcagagcatcggcatcaggcagctgttcaacagcgtgcacctgagagagctgtccgaggccgaagtgaggcggcacagagaggccagacccgccctgctgaccagcaggctgagattcctgcccaagcccagcggcctgaggcccatcgtgaacatggactacatcatgggcgccaggaccttccacagggacaagaaggtgcagcacctgaccagccagctgaaaaccctgttcagcgtgctgaactacgagagggccagaaggcctagcctgctgggcgccagcatgctgggcatggacgacatccacagggcctggcggaccttcgtgctgaggatcagggcccagaaccctgccccccagctgtacttcgtgaaggtggccatcaccggcgcctacgacgccctgcctcaggacagactggtggaggtgatcgccaacgtgatcaggccccaggaaagcacctactgcgtcaggcactacgccgtggtgcagagaaccgccaggggccacgtgaggaaggccttcaagaggcacgtgagcaccttcgccgacctgcagccctacatgaggcagttcgtggagaggctgcaggagaccagcctgctgagggatgccgtggtgatcgagcagagcagcagcctgaacgaggccggcagctccctgttccacctgtttctgaggctggtgcacaaccacgtggtgcggatcggcggcaagagctacatccagtgccagggcgtgcctcagggcagcatcctgagcaccctgctgtgcagcctgtgctacggcgacatggaaaggcggctgttccctggcatcgagcaggacggcgtgctgctgagactggtggacgacttcctgctggtgacccctcatctgacccaggcccaggccttcctgagaaccctggtgaagggcgtgcccgagtacggctgcagggccaacctgcagaaaaccgccgtgaacttccctgtggaggacggcgctctgggatctgctgcccctctgcagctgcctgcccactgcctgttcccttggtgcggcctgctgctggacaccaggaccctggaagtgagctgcgactacagcagctacgcccacaccagcatcagggccagcctgaccttcagccagggcgccaagcccggcaggaacatgcggaggaagctgctggccgtgctgaggctgaagtgctgcgccctgttcctggacctgcaggtcaacggcatccacaccgtgtacatgaacgtgtacaaaatcttcctgctgcaggcctacaggttccacgcctgcgtgctgcagctgcccttcaaccagcccgtgaggaagaaccccagcttcttcctgagggtgatcgccgacaccgccagctgctgctacagcctgctgaaggccagaaatgccggcctgtctctgggagccaagggcgccagcggcctgtttcctagcgaggccgccagatggctgtgcctgcacgcctttctgctgaagctggcccaccacagcggcacctacagatgcctgctgggagccctgcaggccgccaaagcccacctgagcaggcagctgcctagaggaacactggccgccctggaagccgccgctgaccctagcctgaccgccgacttcaagaccatcctggac

SEQ ID NO: 9 - protein, 9 aa, Homo sapiens
APRCRAVRS

SEQ ID NO: 10 - protein, 9 aa, Canis familiaris
APRCRAVRA

SEQ ID NO: 11 - protein, 9 aa, Homo sapiens
EVLPLATFV

SEQ ID NO: 12 - protein, 9 aa, Canis familiaris
EVLPLATFL

SEQ ID NO: 13 - protein, 9 aa, Homo sapiens
VELLRSFFY

SEQ ID NO: 14 - protein, 9 aa, Canis familiaris
VKLLRSFFY

SEQ ID NO: 15 - protein, 9 aa, Homo sapiens
LLTSRLRFI

SEQ ID NO: 16 - protein, 9 aa, Canis familiaris
LLTSRLRFL

SEQ ID NO: 17 - protein, 9 aa, Homo sapiens
RPIVNMDYV

SEQ ID NO: 18 - protein, 9 aa, Canis familiaris
RPIVNMDYI

SEQ ID NO: 19 - protein, 9 aa, Homo sapiens
KALFSVLNY

SEQ ID NO: 20 - protein, 9 aa, Canis familiaris
KTLFSVLNY

SEQ ID NO: 21 - protein, 9 aa, Homo sapiens
GLDDIHRAW

SEQ ID NO: 22 - protein, 9 aa, Canis familiaris
GMDDIHRAW

SEQ ID NO: 23 - protein, 9 aa, Homo sapiens
AWRTFVLRV

SEQ ID NO: 24 - protein, 9 aa, Canis familiaris
AWRTFVLRI

SEQ ID NO: 25 - protein, 9 aa, Homo sapiens
SPLRDAVVI

SEQ ID NO: 26 - protein, 9 aa, Canis familiaris
SLLRDAVVI

SEQ ID NO: 27 - DNA, 69 bp, Artificial Sequence
  nucleotide sequence encoding SEQ ID NO28
atggatgcaatgaagagagggctctgctgtgtgctgctgctgtgtggagcagtcttcgtttcgcccagc

SEQ ID NO: 28 - protein, 23 aa, Artificial Sequence
  Tissue Plasminogen Activator (TPA)
MDAMKRGLCCVLLLCGAVFVSPS

SEQ ID NO: 29 - protein, 1132 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 30 - protein, 1125 aa, Canis familiaris
MPRAPRCRAVRALLRGRYREVLPLATFLRRLGPPGRLLVRRGDPAAFRALVAQCLVCVPWGARPPPAAPCFRQVSCLKELVARVVQRLCERGARNVLAFGFALLDGARGGPPVAFTTSVRSYLPNTVTETLRGSGAWGLLLRRVGDDVLTHLLARCALYLLVAPSCAYQVCGPPLYDLCAPASLPLPAPGLPGLPGLPGLGAGAGASADLRPTRQAQNSGARRRRGSPGSGVPLAKRPRRSVASEPERGAHRSFPRAQQPPVSEAPAVTPAVAASPAASWEGGPPGTRPTTPAWHPYPGPQGVPHDPAHPETKRFLYCSGGRERLRPSFLLSALPPTLSGARKLVETIFLGSAPQKPGAARRMRRLPARYWRMRPLFQELLGNHARCPYRALLRTHCPLRAMAAKEGSGNQAHRGVGICPLERPVAAPQEQTDSTRLVQLLRQHSSPWQVYAFLRACLCWLVPTGLWGSRHNQRRFLRNVKKFISLGKHAKLSLQELTWKMKVRDCTWLHGNPGACCVPAAEHRRREEILARFLVLVDGHIYVVKLLRSFFYVTETTFQKNRLFFYRKSVWSQLQSIGIRQLFNSVHLRELSEAEVRRHREARPALLTSRLRFLPKPSGLRPIVNMDYIMGARTFHRDKKVQHLTSQLKTLFSVLNYERARRPSLLGASMLGMDDIHRAWRTFVLRIRAQNPAPQLYFVKVAITGAYDALPQDRLVEVIANVIRPQESTYCVRHYAVVQRTARGHVRKAFKRHVSTFADLQPYMRQFVERLQETSLLRDAVVIEQSSSLNEAGSSLFHLFLRLVHNHVVRIGGKSYIQCQGVPQGSILSTLLCSLCYGDMERRLFPGIEQDGVLLRLVDDFLLVTPHLTQAQAFLRTLVKGVPEYGCRANLQKTAVNFPVEDGALGSAAPLQLPAHCLFPWCGLLLDTRTLEVSCDYSSYAHTSIRASLTFSQGAKPGRNMRRKLLAVLRLKCCALFLDLQVNGIHTVYMNVYKIFLLQAYRFHACVLQLPFNQPVRKNPSFFLRVIADTASCCYSLLKARNAGLSLGAKGASGLFPSEAARWLCLHAFLLKLAHHSGTYRCLLGALQAAKAHLSRQLPRGTLAALEAAADPSLTADFKTILDSR

SEQ ID NO: 31 - DNA, 498 bp, Artificial Sequence
  nucleotide sequence encoding PFTG
tctagaagcgactgggaccccgtggtgaaggaatggctggtggacaccggctactgctgtgccggcggaatcgccaacgccgaggatggcgtggtgttcgccgctgcagccgacgatgacgacggctggagcaagctgtacaaggacgaccacgaggaggacaccatcggcgaggacggcaacgcctgtggcaaggtgtccatcaacgaggccagcaccatcaaggccgccgtggacgacggcagcgcccccaacggagtgtggatcggcggccagaaatacaaggttgtgaggcccgagaagggcttcgagtacaacgactgtaccttcgacatcaccatgtgtgccagaagcaaaggcggagcccacctgatcaagacccccaacggcagcatcgtgatcgccctgtacgacgaggagaaggagcaggacaagggcaacagcagaaccagcgccctggccttcgccgagtacctgcaccagagcggctactgatga